(19) United States
(12) Patent Application Publication
(10) Pub. No.: US 2004/0236082 A1
(43) Pub. Date: Nov. 25, 2004

(54) Modified staphylococcal enterotoxins and expression systems therefore

(76) Inventors: Matthew J. Marshall, Richland, WA (US); Patrick J. Shiel, Moscow (RU); Philip H. Berger, Apex, NC (US); Gregory A. Bohach, Moscow, ID (US); Carolyn H. Bohach, Moscow, ID (US)

Correspondence Address: Merchant & Gould, PO Box 2903, Minneapolis, MN, 55402-0903, US

(21) Appl. No.: 10/474,171

(22) PCT Filed: Apr. 11, 2002
(86) PCT No.: PCT/US02/11619

Related U.S. Application Data
(60) Provisional application No. 60/283,720, filed on Apr. 13, 2001.

(51) Int. Cl. C07K 14/31

(52) U.S. Cl. 530/395

(57) ABSTRACT

The invention provides mutant pyrogenic toxins. Preferred mutants retain a disulfide loop structure, although the endogenous sequence of the disulfide loop may be modified for example by insertion, deletion and/or substitution of at least one amino acid residue, or by combining a pyrogenic enterotoxin (or a fragment thereof) with another polypeptide to provide a chimeric molecule. Preferred mutants have a disulfide loop having less than about 8 amino acid residues. The invention also provides a system for producing the mutant pyrogenic toxins and methods of use for the mutants.

This application is being filed as a PCT International Patent Application in the name of Idaho Research Foundation, Inc., a U.S. national corporation and U.S. resident, (Applicant for all countries except U.S.); Mathew J. Marshall, a U.S. resident and citizen (Applicant for U.S. only); Patrick J. Shiel, a U.S. resident and citizen (Applicant for U.S. only); Philip H. Berger, a U.S. resident and citizen (Applicant for U.S. only); Gregory A. Bohach, a U.S. resident and citizen (Applicant for U.S. only); and Carolyn H. Bohach, a U.S. resident and citizen (Applicant for U.S. only), on 11 Apr. 2002, designating all countries and claiming priority to U.S. Ser. No. 60/283,720 filed 13 Apr. 2001.

### BACKGROUND OF THE INVENTION

Staphylococcal enterotoxins (SEs) belong to a family of related bacterial Pyrogenic toxins (PTs) produced by Staphylococcus aureus and Streptococcus pyogenes. Staphylococcal PTs include SE types A, B, C1, C2, C3, D, E, G, H, I, J, K, L, M, N, O and P, pyrogenic enterotoxins A and B, and toxic shock syndrome toxin-1 (TSST-1). Streptococcal PTs include streptococcal pyrogenic exotoxins (SPE) A, B, and C, mitogenic factor (MF), streptococcal superantigen (SSA), and the exoproteins recently described from group B, C, F, and G streptococci.

Biological activities common to the pyrogenic toxins include pyrogenicity, enhancement of susceptibility to lethal endotoxic shock, immunosuppression, induction of cytokines, stimulation of lymphocyte proliferation, and superantigenicity. These biological activities have been linked to pathogenesis of the potentially fatal diseases Toxic Shock Syndrome (TSS) and TSS-like illness. Many characteristic symptoms associated with PT-induced disease have been linked to the ability of these toxins to stimulate a large percentage of T-cells via a mechanism not requiring typical antigen presentation. This type of stimulatory ability is known as superantigenicity. SEs also have a unique ability to induce emesis, and have been shown to be a causative agent of staphylococcal food poising (SFP). This biological property distinguishes the SEs from the other PTs.

Toxins of the pyrogenic toxin family are 22-28 kDa monomeric proteins, which share a significant amount of amino acid sequence homology. Although the level of primary sequence homology varies between members of the family, many of the conserved residues have been found to be located in four primary sequence regions. These regions are presumed to be involved with the shared biological activities found within this toxin family. Additionally, SEs possess two cysteine residues, separated by a short stretch of amino acids that are covalently linked through the formation of a disulfide bond to form a characteristic disulfide loop structure unique to the SEs.

### SUMMARY OF THE INVENTION

The invention provides modified staphylococcal pyrogenic toxins. Preferred mutants retain a disulfide loop structure, although the endogenous sequence of the disulfide loop may be modified, for example by insertion, deletion and/or substitution of at least one amino acid residue, or by combining a pyrogenic toxin (or a fragment thereof) with another polypeptide to provide a chimeric molecule. Preferred mutants have a disulfide loop having less than about 5 amino acid residues that have reduced toxicity.

The invention also provides a system for producing the modified staphylococcal enterotoxins of the invention. A preferred system includes the use of a plant host cell, most preferably plant tissues from Nicotiana benthamiana, Chenopodium quinoa, Nicotiana tabacum, Solanum tuberosum, or Licopersicon esuclentum.

The invention also provides methods of use for the modified staphylococcal enterotoxins of the invention. One preferred use is as a vaccine to protect against diseases such as toxic shock syndrome and food poisoning.

### BRIEF DESCRIPTION OF THE FIGURES

FIG. 1 is a table showing the SEC1 mutants generated by a combination of PCR and exonuclease-mediated alteration and confirmed by DNA sequencing.

FIG. 2 is a table showing the calculated molecular weights (in Daltons) of the six SEC deletion mutants.

FIG. 3 is a picture of a 12.5% SDS-PAGE of SEC1 deletion mutants. SEC1 and SEC1 mutant toxins, designated at the bottom, were able to be clearly distinguished. Pre-stained molecular weight markers are shown at the far left.

FIG. 4 is a gel showing the trypsin lability of SEC1 and SEC1 mutant toxins. Purified toxin (1 μg/μl) was incubated in the presence of trypsin (80 μg/ml) at 37° C. Following various digestion time points (top), samples were removed and analyzed by SDS-PAGE.

FIG. 5 is a gel showing the pepsin lability of SEC1 and SEC1 mutant toxins. Purified toxin (1 μg/μl) was incubated in the presence of pepsin (500 μg/ml) at 37° C. Following various digestion time points (top), samples were removed and analyzed by SDS-PAGE.

FIGS. 6A and 6B are photographs of gels showing the relative in vitro degradation rates of SEC1 and SEC1 mutants in gastric fluid. Purified toxin (1 μg/μl) was digested at 37° C. in diluted gastric, fluid (1:2 in physiological saline). Following various digestion time points (top), samples were removed and analyzed by SDS-PAGE. A. One hour digestion of loop deletion mutants. B. Four hour digestion of loop deletion mutants.

FIG. 7 is a graph showing the free sulfhydryl in SEC1 mutant toxins. Each mutant, indicated below, was assayed under non-reducing conditions (clear bars) and under reducing conditions (shaded bars). Data indicate the average of at least three experimental runs, and measured as number of free sulfhydryl residues/toxin molecule (left) and absorbance at 412 nm±the standard error of the mean (right).

FIG. 8 is a graph comparing T-cell proliferation induced by SEC1 and SEC1 mutants. Enriched human T-cells were incubated for 4 days, in the presence of a single toxin, over a log range of concentrations shown on a log scale. Proliferation was expressed in counts per minute (CPM) of ³H-thymidine incorporation into cellular DNA, ± the standard error of the mean.

FIG. 9 is a table showing the emetic response induced by SEC1 loop deletion mutants. Results are expressed as Number of animals exhibiting emesis/Total number of animals. Toxin dose for each kg of animal body weight is indicated to the left. All experimental animals were observed for at least 12 hours for an emetic response. “--” indicates: dose response was not determined. A. Experimental results obtained using a modification of the standard monkey feeding assay (Chang et al. (1979) Mol Gen Genet. 168(1):111-5) B. Experimental results obtained using the syringe-feeding assay.

FIGS. 10A and 10B are tables showing the in vivo pyrogenic response and enhancement of shock susceptibility induced by SEC1 and SEC1 mutants in a rabbit model. Native and mutant toxin doses, listed at left, indicate toxin dose intravenously injected for each kg of animal body weight. A. The mean rectal temperature rise (° C.) following intravenous administration of enterotoxin. B. Number of experimental animals exhibiting enhanced endotoxic shock lethality/total number of animals. Endotoxin (10 μg/kg) was administered intravenously four hours following initial enterotoxin dose.

FIG. 11 is a table showing the in vivo protection of rabbits immunized with the SEC1-12“C” mutant against pyrogenic response and enhancement of shock susceptibility induced by SEC1. Rabbits were challenged with 5 μg/kg of biologically active SEC1. Endotoxin (10 μg/kg) was administered intravenously four hours following initial enterotoxin dose. Survival indicates immunity to the enhancement of lethal endotoxic shock by SEC1.

FIG. 12 is a schematic showing the construction of the recombinant 30B.SEC1-12C. An illustration of the infectious TMV-based vector, TMV-30B and the wild type TMV strain, U1, from which it was derived. The SEC1-12“C” gene was inserted into a PmeI site located within the multiple cloning sites (MCS). Arrows (⇄) indicate the strain of the virus used as described in the text. Boxes represent viral genes and lines indicate nontranslated sequences. RdRp, TMV RNA dependent RNA polymerase; MP, TMV movement protein; CP, TMV coat protein; *, indicates subgenomic promoters. Other important features shown include the location of the T7 RNA polymerase promoter and the KpnI site.

FIG. 13 is a table showing 30B.GFP host range and reporter gene expression.

FIG. 14 is a photograph of a gel showing a Western blot analysis of Chenopodium quinoa plants infected with 30B.SEC1-12“C”. (A) Western blot analysis of soluble proteins isolated from leaves at 10 days post inoculation. Lane 1, Extract from plant infected with 30B.SEC1-12“C”; lane 2, extract from plant infected with TMV-30B; lane 3, extract from mock-inoculated plant containing no virus. Molecular weights (kDa) are indicated at right. (B) Time course experiment showing the time dependent expression of the SEC1-12“C”. Leaf tissue was harvested at days 0, 3, 5, 7, 9, 10, 11, and 13 post inoculation and analyzed with western blot. Mock, extract from mock-inoculated plant containing no virus; MW-STD, Benchmark™ pre-stained molecular weight markers (GibbCo-BRL) (kDa).

FIG. 15 is a table showing the in vivo protection of rabbits immunized with Chenopodium quinoa produced SEC1-12C against challenge with biologically active SEC1. Rabbits were challenged with 5 μg/kg of biologically active SEC1. Endotoxin (10 μg/kg) was administered intravenously four hours following initial enterotoxin dose. Survival indicates protection to the enhancement of lethal endotoxic shock by SEC1.

### DETAILED DESCRIPTION

### I. Pyrogenic Toxins (PTs)

Pyrogenic toxins (PTs) constitute a family of exotoxins produced by species of gram positive cocci, such as Staphylococcus and Streptococcus. The PTs are characterized by shared ability to induce fever, enhance host susceptibility to endotoxin shock, and induce T cell proliferation through action as superantigens. Examples of PTs include TSST-1, staphylococcal enterotoxins (SEs), and streptococcal pyrogenic exotoxins (SPEs). In addition to the activities listed above, some PTs have additional activities that are not shared by all PTs. For example, the staphylococcal enterotoxins (SEs) induce emesis and diarrhea when ingested. Structurally, the PTs have varying degrees of relatedness at the amino acid and nucleotide sequence levels. A number of the PTs include a disulfide loop as a structural feature. The SEs have a disulfide loop, as do some others in this family. Examples of other PTs that have a disulfide loop are the streptococcal superantigen (“SSA”) and streptococcal pyrogenic exotoxin A (“SPEA”).

The enterotoxins of Staphylococcus aureus form a group of serologically distinct proteins. These proteins were originally recognized as the causative agents of staphylococcal food poisoning. Ingestion of preformed enterotoxin in contaminated food leads to the rapid development (within two to six hours) of symptoms of vomiting and diarrhea that are characteristic of staphylococcal food poisoning. Toxic shock syndrome toxin-1, TSST-1, a distantly related protein also produced by S. aureus, is classically responsible for the toxic shock syndrome, although other SEs may result in the syndrome due to the induction of cytokines.

Enterotoxins produced by Staphylococcus aureus include a group of related proteins of about 20 to 30 Kd. The complete amino acid composition of a number of SEs and streptococcal pyrogenic exotoxin has been reported (see e.g., PCT Patent Appl. No. WO 93/24136, the disclosure of which is hereby incorporated by reference herein in its entirety).

Staphylococcal enterotoxins (“SEs”) were initially classified on the basis of their antigenic properties into groups A, B, C1, C2, C3, D, and E. Subsequent relatedness was based on peptide and DNA sequence data. Among the SEs, groups B and C are closely related and groups A, D, and E are closely related in amino acid sequence. SEC1, SEC2, and SEC3 and related isolates share approximately 95% sequence similarity. Table 1 shows the alignment of the predicted sequences of the eight known SEC variants following cleavage of the signal peptide. Amino acid positions that contain residues that are not conserved among these SEC variants are indicated by asterisks. SEB and SEC are approximately 45-50% homologous. In contrast, non-enterotoxin superantigens, TSST-1 and Streptococcal Pyrogenic Enterotoxin C (SPEC) share only approximately 20% primary sequence homology to SEC. Despite these differences, the tertiary structure of the various enterotoxins show nearly identical folds.

The SEs A, B, C₁, C₂, C₃, D, E, G and H share a common structural feature of a disulfide bond not present in many other pyrogenic toxins. Table 2 shows the position of the disulfide bond in a number of enterotoxins. Sequence data demonstrate a high degree of similarity in four regions of the enterotoxins (Table 3). The peptides implicated in potential receptor binding correspond to regions 1 and 3, which form a groove in the molecule. Amino acid residues within and adjacent to the α₃ cavity of SEC3 have been shown to relate to T-cell activation.

SEs, aside from the associated acute gastroenteritis and toxic shock syndrome, have a variety of potential beneficial biological effects. The biological effects of these agents and the toxic shock syndrome toxin are due in part to the ability of SEs to induce cytokines, including IL-1, IL-2, and tumor necrosis factor (“TNF”). More recently SEB and toxic shock syndrome toxin (“TSST-1”) have been shown to induce interleukin-12, an inducer of cell-mediated immunity, in human peripheral blood mononuclear cells. (See Leung et al., J Exp Med, 181:747 (1995)). The antitumor activity in rabbits using 40 to 60 μg/kg of a SE is disclosed in PCT Patent Appl. Nos. WO 91/10680 and WO 93/24136.

In contrast to other species, man is extremely sensitive to enterotoxins. One (1) mg of TSST-1, approximately 15 nanogram/kg, can be lethal. Therefore, the recommended doses currently proposed in the art for treating man are unacceptable. There is a need, therefore, for mutant SEs that are non-toxic at anticipated doses for man while retaining desirable biological activity.

### II. Mutant Enterotoxins

Because of the sensitivity of man to enterotoxins, it may be desirable to create SE mutants that are at least 1000-fold, or more, less toxic compared to native enterotoxins. However, it is important that the mutant enterotoxins retain at least some (i.e., at least 1% to 10%) of the beneficial biological activities of the native enterotoxin, such as immune cell stimulation, cytokine activity and antigen activity. As used herein, the terms “toxic” and “toxicity” refer to the ability to induce or enhance fever or shock systemically or gastroenteritis if ingested. Other examples of a toxic response include emesis, pyrogenesis, and mitogenesis. The term “lethal” refers to the induction of lethal shock in a well-characterized animal model or toxic shock syndrome. The term “biological activity” refers to both beneficial and detrimental activities. The biological effects of SE toxins appear to be related to the structural stability of the toxin. Alterations in the native structure of the toxin may affect protein stability and reduce the ability to induce the biological activities associated with these toxins.

Modified or mutant enterotoxins with reduced toxicity are known. As used herein, the term “reduced toxicity” means the toxin induces a reduced emetic and/or pyrogenic response and/or lethal shock enhancement in comparison to the wild-type toxin. Preferably, the emetic and/or pyrogenic response is reduce by at least about 100-fold. Examples of mutants with reduced toxicities include, carboxymethylated SEB, which displays a loss of gastrointestinal toxicity but not mitogenic activity. One active site of TSST-1 is between amino acids residue 115 and 141—point mutation of site 135 from histidine to alanine results in a loss of mitogenic activity and toxicity (See Bonventre P. F., et al. Infect Immun 63:509 (1995)). The disulfide bond of SEC1 between residues 93 and 110 does not appear to be directly required for activity, but affects acvitiy indirectly by stabilizing protine structure (See Hovde et al., Mol Microbiol 13:897 (1994)). Generally, mutants of SEC1, which are unable to form a native structure in the area of the disulfide bond, are about ten times less toxic than the native toxin while retaining biological activity. (See e.g., Hovde et al., Molec Microbiol 13:897 (1994)).

The invention provides modified PTs, such as SEs, which have reduced toxicity. As used herein, the term “Staphylococcal enterotoxin” or “SE” refers to the full-length, wild-type protein, and mutant proteins, as well as fragments of a wild-type or mutant peptide. The terms “peptide” and “protein” are used interchangeably herein. The term “full-length” peptide refers to the peptide encoded by the full DNA coding sequence. For example, DNA sequences encoding full-length SE proteins are known, as are the corresponding full-length amino acid sequences (See, e.g., PCT Patent Appl. No. WO 93/24136, the disclosure of which is hereby incorporated by reference in its entirety). A full-length peptide can be either a wild-type or a mutant peptide. The term “wild-type” refers to a naturally occurring phenotype that is characteristic of most of the members of a species with the gene in question (in contrast to the phenotype of a mutant). The term “mutant” and “modified” are used interchangeably and refer to a peptide or protein not having a wild-type sequence. The term “mutein” refers to a mutant protein produced by site-specific mutagenesis or other recombinant DNA technique wherein the mutein retains some of the desired activity of the peptide. The term “fragment” refers to a sequence that includes at least part of the wild-type sequence or mutant sequence, wherein the fragment retains the desired activity of the peptide.

Preferred fragments and mutants retain amino acid residues within the disulfide loop, although the sequence of the disulfide loop may be truncated. Preferably, the DNA or RNA encoding the fragment or mutant is capable of hybridizing to all or a portion of the DNA or RNA encoding a wild-type SE protein, or its complement, under stringent or moderately stringent hybridization conditions (as defined herein).

The term “hybridizing” refers to the pairing of complementary nucleic acids. Hybridization” can include hydrogen bonding, which may be Watson-Crick, Hoogsteen or reversed Hoogsteen hydrogen bonding, between complementary nucleoside or nucleotide bases. Hybridization and the strength of hybridization (i.e., the strength of the association between the nucleic acids) is influenced by such factors as the degree of complementarity between the nucleic acids, stringency of the conditions involved, the melting temperature (Tₘ) of the formed hybrid, and the G:C ratio within the nucleic acids. Complementarity may be “partial,” in which only some of the bases of the nucleic acids are matched according to the base pairing rules. Alternatively, there may be “complete” or “total” complementarity between the nucleic acids. The degree of complementarity between the nucleic acid strands has effects on the efficiency and strength of hybridization between the nucleic acid strands.

Preferably, the hybridizing portion of the hybridizing nucleic acids is at least 15 (e.g., 20, 25, 30 or 50) nucleotides in length and at least 80% (e.g., at least 90%, 95%, or 98%) identical to a sequence of a wild type SE, or its complement, or fragments thereof.

As used herein, the term “percent homology” or “percent identity” of two nucleic acid sequences is determined using the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877. Such an algorithm is incorporated into the NBLAST program of Altschul et al. (1990) J Mol. BiOl. 215: 402-410. To obtain gapped alignments for comparision purposes, Gapped BLAST is used as described by Altschul et al. (1997) Nuelic Acids Res. 25: 3389-3402. When using BLAST and gapped BLAST programs, the default parameters of the respective programs (e.g., NBLAST) are used.

Examples of native SE which can be modified to form the present low toxicity toxins include type A, B, C, D, E, G, and H SEs. Type C staphylococcal enterotoxins such as staphylococcal enterotoxin C1, staphylococcal enterotoxin C2, staphylococcal enterotoxin C2, staphylococcal enterotoxin C-MNCopeland, staphylococcal enterotoxin C-4446, staphylococcal enterotoxin C-bovine (GenBank Accession No. L13374), staphylococcal enterotoxin C-canine (GenBank Accession No. V19526) and staphylococcal enterotoxin C-ovine (GenBank Accession No. L13379) are particularly suitable enterotoxins for modification by deletion of a portion of the disulfide loop region to form a staphylococcal enterotoxin with decreased toxicity.

As discussed above, most, but not all, native SEs have a disulfide loop. The terms “disulfide loop” and “disulfide loop region” are used interchangeably herein. As employed in this application, these terms refer to the sequence of about 10 to about 30 amino acid residues forming a loop defined by a disulfide bond in a native pyrogenic toxin. The term “disulfide loop region” also refers to the corresponding portion of the sequence of a modified pyrogenic toxin that has been produced by deletion, substitution or addition of one or more amino acid residues of the disulfide loop of a native pyrogenic toxin or of the two cysteines responsible for its formation. The disulfide loop region is defined to begin with the N-terminal Cys residue and end with the C-terminal Cys residue of the loop, e.g., amino acid residues 93-110 of staphylococcal enterotoxin C1 or resides substituted at these positions. As used herein, the positions of the disulfide loop region for a given native pyrogenic toxin are numbered beginning with the N-terminal cyteine residue in the loop, e.g., position 93 of type B or C staphylococcal enterotoxins is also referred to herein as position 1 of the disulfide loop region. Generally, the loop size of the SE toxin correlates with stability that affects both toxicity and biological activity of the mutant, with a larger loop (i.e., between about 16 and 20 amino acid residues, preferably about 18) having more stability than a smaller loop (i.e., between about 10 to 12, preferably about 11 amino acid residues).

Preferred SE mutants of the invention include deletions, substitutions and/or insertions of amino acids from within the disulfide bond loop. The modification of the disulfide loop typically includes deletion of at least about 25% to 95% of the amino acid residues within the disulfide loop. This typically results in the deletion of between about 4 to 18 amino acid residues from the disulfide loop region. Preferably, the modified disulfide loop region contains no more than about 8 amino acid residues, preferably no more than 3 amino acid residues. The phrase “amino acid residues within the disulfide loop” refers to the number of amino acids between (i.e., not including) the two cysteine residues forming the disulfide bond. The most preferred mutants retain an intermolecular disulfide loop structure.

In other mutants, an exogenous sequence of one or more amino acids can be inserted into the peptides sequence, preferably within the disulfide loop. More preferably, an exogenous sequence of one or more non-native amino acids is inserted within the disulfide loop in combination with a deletion of one or more of amino acids from the wild-type sequence. As used herein, the term “exogenous” is intended to refer to amino acids that are not found within the endogenous SE sequence as it exists in nature. Preferably, the exogenous sequence contains from 1 to 30 amino acid residues, more preferably between 3 and 15 amino acid residues. In one embodiment, the exogenous sequence contains a sequence of between 1 and 30 alanine residues. Another preferred residue is glycine.

These mutants focus on interfering with the interaction between the toxin and receptors. The disulfide loop is near the receptor-binding site for both T cells and MHC II. Also, the structure around the disulfide loop influences the emetic response.

Suitable mutants also include mutants having one or more conservative amino acid substitutions, either within the disulfide loop or outside of the loop. As used herein, “conservative amino acid substitution” refers to a replacement of one or more amino acid residue with a different residue having a sidechain with at least one similar biochemical characteristic, such as size, shape, charge or polarity. Preferably, the substitution impacts receptor binding and/or toxicity.

Other suitable mutants include chimeric molecules. As used herein, the term “chimera” refers to hybrid molecules that contain at least a fragment of a SE amino acid sequence operably connected to a heterologous polypeptide or amino acid sequence. For example, an N-terminal sequence from one SE (e.g., SEC1, or any other SE) can be combined with a C-terminal sequence from another SE (e.g., SEA, or any other SE), or vice versa, to form a chimera. A chimera provides a molecule that has antigenic and/or biological properties of two or more toxins. As used herein, the terms “N-terminus” or “N-terminal sequence” refer to the amino acid sequence of the N-terminal globular domain. Likewise, the terms “C-terminus” or “C-terminal sequence” refer to the sequence of amino acids of the C-terminal globular domain. The two domains are separated generally by amino acid residues 112-130 (as numbered by Hoffmann et al (1994) Infect Immun. 62:3396-3407). The two globular domains are visually apparent when viewing a model of the protein. Other chimeras may include a fragment or a full length SE sequence in combination with an antibody. Generally, the first 10-20 amino acid residues can be deleted from the N-terminal sequence without affecting protein activity, more preferably the first 14-15 amino acids.

### III. Mutagenesis

The mutant enterotoxins sequences can be prepared by methods known in the art. Typically, a mutant staphylococcal enterotoxin is generated by genetic alteration of an oligonucleotide sequence encoding the SE. As used herein, the term “oligonucleotide” or “nucleic acid sequence” refers to an oligomer or polymer of ribonucleic acid (RNA) or deoxyribonucleic acid (DNA) or mimetics thereof. As used herein, the term “isolated nucleic acid sequence” refers to a nucleic acid, including both DNA and/or RNA, which in some way is not identical to that of any naturally occurring nucleic acid or to that of any naturally occurring genomic nucleic acid. The term therefore covers, for example, (a) DNA that has the sequence of part of a naturally occurring genomic DNA molecule, but is not flanked by both of the coding sequences that flank the DNA in the genome of the organism in which it naturally occurs; (b) a nucleic acid incorporated into a vector or into the genomic DNA of a prokaryote or eukaryote in a manner such that that resulting molecule is not identical to any naturally occurring vector or genomic DNA; (c) a separate molecule such as a cDNA, a genomic fragment, a fragment (either DNA or RNA) produced by polymerase chain reaction (PCR), or a restriction fragment; and (d) a recombinant nucleic acid sequence that is part of a hybrid gene (i.e., a gene encoding a fusion protein). The term “isolated” may also be used interchangeably with the term “purified.”

As used herein, the terms “complementary” or “complement”, when used in reference to a nucleic acid sequence, refers to sequences that are related by the base-pairing rules developed by Watson and Crick. For example, for the sequence “T-G-A” the complementary sequence is “A-C-T.”

The invention also includes nucleic acid sequences that are capable of hybridizing to all or a portion of a nucleic acid sequence encoding a staphylococcal enterotoxin, or its complement, under stringent or moderately stringent hybridization conditions (as defined herein). The term “hybridizing” refers to the pairing of complementary nucleic acids. Hybridization” can include hydrogen bonding, which may be Watson-Crick, Hoogsteen or reversed Hoogsteen hydrogen bonding, between complementary nucleoside or nucleotide bases. Hybridization and the strength of hybridization (i.e., the strength of the association between the nucleic acids) is influenced by such factors as the degree of complementarity between the nucleic acids, stringency of the conditions involved, the melting temperature (Tₘ) of the formed hybrid, and the G:C ratio within the nucleic acids. Complementarity may be “partial,” in which only some of the bases of the nucleic acids are matched according to the base pairing rules. Alternatively, there may be “complete” or “total” complementarity between the nucleic acids. The degree of complementarity between the nucleic acid strands has effects on the efficiency and strength of hybridization between the nucleic acid strands. If desired, the hybridizing sequence can include a label, such as a radiolabel (e.g., ³H, ¹⁴C, ³²P or ¹²⁵I, etc.) or a fluorescent label (e.g., fluorescein, rhodamine, etc.).

The hybridizing portion of the hybridizing nucleic acids is typically at least 15 (e.g., 20, 25, 30 or 50) nucleotides in length and at least 80% (e.g., at least 95% or at least 98%) identical to a wild-type sequence encoding an SE, or its complement. Hybridizing nucleic acids of the type described herein can be used, for example, as a cloning probe, a primer (e.g., a PCR primer), or a diagnostic probe. Hybridization of the oligonucleotide probe to a nucleic acid sample typically is performed under stringent conditions.

Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentrion of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C. In practice, the change in Tm can be between 0.5° C. and 1.5° C. per 1% mismatch. As used herein, “stringent conditions” involve hybridizing at 68° C. in 5×SSC/5×Denhardt's solution/1.0% SDS, and washing in 0.2×SSC/0.1% SDS at room temperature. “Moderately stringent” conditions include washing in 3×SSC at 42° C. The parameters of salt concentration and temperature can be varied to achieve the optimal level of identity between the probe and the target nucleic acid. Additional guidance regarding such conditions is readily available, for example, by Sambrook et al, 1989, Molecular Clonging, A Laboratory Manual, Cold Spring Harbor Press, N.Y.

As used herein, the term “percent homology” or “percent identity” of two nucleic acid sequences is determined using the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877. Such an algorithm is incorporated into the NBLAST program of Altschul et al. (1990) J. Mol. BiOl. 215: 402-410. To obtain gapped alignments for comparision purposes, Gapped BLAST is used as described by Altschul et al. (1997) Nuelic Acids Res. 25: 3389-3402. When using BLAST and gapped BLAST programs, the default parameters of the respective programs (e.g., NBLAST) are used.

The invention also includes degenerate variants of wild-type nucleic acid sequences encoding SEs. The genetic code is made up of sixty-four codons. Three code for chain termination. The remaining sixty-one triplets encode the twenty amino acids. Many amino acids are coded by more than one codon. Thus, the genetic code is said to be degenerate. A “degenerate variant” refers to a nucleic acid sequence in which a codon in the nucleic acid sequence, which codes for a particular amino acid, is exchanged for another codon that codes for the same amino acid. For example, in a degenerate variant, the sequence ACU, coding for threonine, may be exchanged for the sequence ACC, which also codes for threonine. See, for example, Stryer, (1988) Biochemistry, W.H. Freeman and Co., New York, Chapter 5, page 107, Table 5.5. If desired, one or more such exchanges can be made in a degenerate variant.

### IV. Vectors

The invention also includes expression vectors containing a nucleic acid sequence encoding a mutant SE. As used herein, the term “expression vector” refers to a construct containing a nucleic acid sequence that is operably linked to a suitable control sequence capable of effecting expression of the nucleic acid sequence in a suitable host.

Nucleic acid is “operably linked” when it is placed into a functional relationship with another nucleic acid sequence. For example, DNA sequence encoding a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in some cases, contiguous and in reading phase. However, some sequences, such as enhancers, do not have to be contiguous. Linking is accomplished by ligation at convenient restriction sites. If such sites do not exist, the synthetic oligonucleotide adaptors or linkers are used in accordance with conventional practice.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The nucleic acid (e.g., cDNA or genomic DNA) encoding mutant SE may be inserted into a replicable vector for cloning (amplification of the DNA) or for expression. As used herein, “expression vector” means a DNA construct including a DNA sequence (e.g., a sequence encoding a fluorescent protein) that is operably linked to a suitable control sequence (e.g. all or part of a mutagen sensitive gene) capable of affecting the expression of the DNA in a suitable host. Such control sequences may include a promoter to affect transcription, an optional operator sequence to control transcription, a sequence encoding suitable ribosome-binding sites on the mRNA, and sequences that control termination of transcription and translation. Different cell types may be employed with different expression vectors. The vector may be a plasmid, a phage particle, or simply a potential genomic insert. Once transformed into a suitable host, the vector may replicate and function independently of the host genome, or may, under suitable conditions, integrate into the genome itself. In the present specification, plasmid and vector are sometimes used interchangeably. However, the invention is intended to include other forms of expression vectors that serve equivalent functions and which are, or become, known in the art. Useful expression vectors, for example, can include segments of chromosomal, non-chromosomal and synthetic DNA sequences such as various known derivatives of known bacterial plasmids, e.g., plasmids from E. coli including Co1 E1, pCR1, pBR322, pMb9, pUC 19 and their derivatives, wider host range plasmids, e.g., RP4, phage DNAs e.g., the numerous derivatives of phage 11, e.g., NM989, and other DNA phages, e.g., M13 and filamentous single stranded DNA phages, yeast plasmids such as the 2 mm plasmid or derivatives thereof, vectors useful in eukaryotic cells, such as vectors useful in animal cells and vectors derived from combinations of plasmids and phage DNAs, such as plasmids which have been modified to employ phage DNA or other expression control sequences. Other suitable vectors include viral vectors based on Adeno Associated Virus (AAV) serotypes and viral vectors with adenovirus, retrovirus, and as chimeric virus backbones, e.g., adeno-retroviral or retro-adenoviral vectors. A particularly preferred vector is a recombinant tobacco mosaic virus (TMV) vector.

Both expression and cloning vectors contain a nucleic acid sequence that enables the vector to replicate in one or more selected host cells. Such sequences are well known for a variety of bacteria, yeast, and viruses.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacillus.

Expression vectors used in eukaryotic host cells (yeast, fungi, insect, plant, animal, human, or nucleated cells from other multicellular organisms) may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the mutant SE protein.

Expression techniques using the expression vectors of the present invention are known in the art and are described generally in, for example, Sambrook et al., Molecular Cloning: A Laboratory Manual, Second Edition, Cold Spring Harbor Press (1989).

### V. Production of Mutant SE

Mutant SE can be produced by culturing cells transformed or transfected with a vector containing a nucleic acid encoding the mutant SE. Mutant SE, or portions thereof, may also be produced by direct peptide synthesis using solid-phase techniques (see, e.g., Stewart et al., Solid-Phase Peptide Synthesis, W.H. Freeman Co., San Francisco, Calif. (1969); Merrifield, J. Am. Chem. Soc., 85: 2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the mutant SE protein may be chemically synthesized separately and combined using chemical or enzymatic methods to produce the full-length mutant SE.

Typically, host cells are transfected or transformed with expression or cloning vectors described herein for mutant SE production and cultured in conventional nutrient media modified as appropriate for inducing promoters, selecting transformants, or amplifying the genes encoding the desired sequences. Culture conditions, such as media, temperature, and pH, can be selected by the skilled artisan without undue experimentation.

Methods of transfection are known, for example, CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Other transfection methods include protoplast transformation for Staphylococcus as described by Chang and Cohen, Molecular and General Genetics, 168:111-115 (1979).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli and Staphylococcus aureus. In addition to prokaryotes, eukaryotic microbes such as filamentous fungi or yeast are suitable cloning or expression hosts for mutant SE-encoding vectors.

Typically proteins, such as staphylococcal enterotoxins, are produced using a microorganism culture, such as a bacterial culture. However, the use of a transgenic plant may be more desirable because a transgenic plant system can provide increased levels of recombinant protein expression, protein stability, and post-translational modification. Additionally, tissue-specific promoters and plant-optimized synthetic genes can be used to increase expression levels and enhance subunit oligomerization. However, recombinant protein expression levels obtained in most plant systems are not sufficient as a replacement for traditional vaccine production schemes.

The invention also provides a high-level expression system in plant tissue. Generally, edible plants are preferred hosts. Most preferred plant tissues include tissues from Nicotiana benthamiana, Chenopodium quinoa, Nicotiana tabacum, Solanum tuberosum, or Licopersicon esuclentum.

### VI. Purification of Mutant SE

Mutant SE may be recovered from culture medium or from host cell lysates. If membrane-bound, it can be released from the membrane using a suitable detergent solution (e.g. Triton-X 100) or by enzymatic cleavage. Cells employed in expression of mutant SE can be disrupted by various physical or chemical means, such as freeze-thaw cycling, sonication, mechanical disruption, or cell lysing agents.

It may be desired to purify SE from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the mutant SE. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular mutant SE produced.

### VII. Pharmaceutical Compositions and Uses

The invention provides a method for enhancing immune function nonspecifically and for vaccination against staphylococcal food poisoning. The beneficial biological effects are due in part to the ability of SEs to activate leukocytes and induce cytokines. The mutant SE can be used in human as well as veterinary applications. For such purposes, the mutant SE can be employed in pharmaceutical compositions, containing one or more active ingredients plus one or more pharmaceutically acceptable carriers, diluents, fillers, binders and other excipients, depending upon the mode of administration and dosage form contemplated.

The peptide may be delivered to the patient by methods known in the field for delivery of peptide therapeutic agents. Preferably, to provide protection from food poisoning, the SE mutant is mixed with a delivery vehicle and administered orally, for example, as an “edible vaccine.” The composition typically contains a pharmaceutically acceptable carrier mixed with the agent and other components in the pharmaceutical composition. By “pharmaceutically acceptable carrier” is intended a carrier that is conventionally used in the art to facilitate the storage, administration, and/or the healing effect of the agent. A carrier may also reduce any undesirable side effects of the agent. A suitable carrier should be stable, i.e., incapable of reacting with other ingredients in the formulation. It should not produce significant local or systemic adverse effect in recipients at the dosages and concentrations employed for treatment. Such carriers are generally known in the art.

Therapeutic compositions of the SE mutant can be prepared by mixing the desired molecule having the appropriate degree of purity with optional pharmaceutically acceptable carriers, excipients, or stabilizers (Remington's Pharmaceutical Sciences, 16th edition, Oslo, A. ed. (1980)), in the form of lyophilized formulations or aqueous solutions. Acceptable carriers, excipients, or stabilizers are preferably nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g.; Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

SE mutant protein to be used for in vivo administration should be sterile. This is readily accomplished by filtration through sterile filtration membranes, prior to or following lyophilization and reconstitution. Therapeutic peptide compositions generally are placed into a container having a sterile access port, for example, an intravenous solution bag or vial having a stopper pierceable by a hypodermic injection needle. The formulations are preferably administered as repeated intravenous (i.v.), subcutaneous (s.c.), or intramuscular (i.m.) injections, or as aerosol formulations suitable for intranasal or intrapulmonary delivery (for intrapulmonary delivery see, e.g., EP 257,956).

SE mutant peptide can also be administered in the form of sustained-released preparations. Suitable examples of sustained-release preparations include semipermeable matrices of solid hydrophobic polymers containing the protein, which matrices are in the form of shaped articles, e.g., films, or microcapsules.

The therapeutically effective dose of SE mutant peptide will, of course, vary depending on such factors as the intended therapy, the pathological condition to be treated, the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect.

The route of administration of SE mutant is in accord with known methods, e.g., by injection or infusion by intravenous, intramuscular, intracerebral, intraperitoneal, intracerobrospinal, subcutaneous, parenteral, intraocular, intraarticular, intrasynovial, intrathecal, oral, topical, or inhalation routes, or by sustained-release systems. The SE mutant is suitably administered by intratumoral, peritumoral, intralesional, or perilesional routes, to exert local as well as systemic therapeutic effects. The SE mutant can be administered in combination with (serially or simultaneously) another agent that is effective for those purposes, either in the same composition or as separate compositions. For example, the SE mutant can be administered in an amount between about 1 μg/kg to 1000 μg/kg body weight.

Additionally, mutant SE toxin can be used as a vaccine to help reduce or prevent biological effects associated with toxic shock syndrome. Previous studies have reported that immunity to SE biological activity can be developed following repeated injection into an animal model (Bohach et al. (1988) Infect Immun. 56(2):400-4; Schlievert, P. M. (1982) Infect Immun. 36(1):123-8), and many of the associated disease symptoms have been linked to cytokine induction following T-cell stimulation (Bohach et al. (1996) “The staphylococcal and streptococcal pyrogenic toxin family.”, In B. R. Singh and A. T. Tu (ed.), Natural Toxins II. Plenum Press, New York., p. 131-154).

### WORKING EXAMPLES

The invention will be further described by reference to the following examples. These examples illustrate but do not limit the scope of the invention that has been set forth herein. Variation within the concepts of the invention will be apparent.

### I. Mutant SEC1

Six mutant SEC1 toxins containing sequential deletions within the loop region were generated. Each mutant was then evaluated for its ability to resist proteolytic degradation and to induce the biological activities associated with wild type SEC1.

### Example 1

### Generation of Mutant SEC1

A. Native SEC1

The structural gene for SEC1 from S. aureus strain MNDON (Bohach et al. (1987) Infect Immun. 55(2):428-32) was used as native SEC1. A 1.0 Kb HindIII-BamHI (3′-5′) fragment containing sec⁺_{mndon}, was sub-cloned from pMIN146 into the multiple cloning site of the 5.6 Kb pALTER™-1 phagemid vector. This vector was then used to transform E. coli TG1. Mutagenesis was performed on sec⁺_{mndon} obtained from E. coli TG1.

B. Mutagenesis

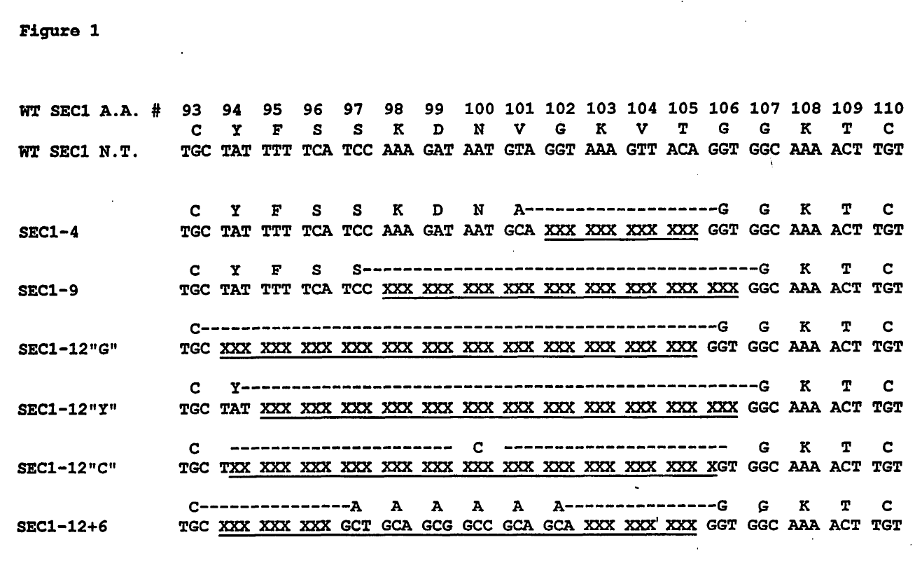

Native SEC1 has two cysteine residues located at positions 93 and 110 of the primary sequence. The cysteine residues are involved in the formation of a disulfide bond that produces a loop region (FIG. 1). To study the involvement of the loop region in the biological activities of SEs; mutant SE toxins were generated with various alterations within the loop region.

M13 helper phage (Stratagene, La Jolla Calif.) was utilized to generate a single stranded template for the mutagenesis reaction. Site directed mutagenesis procedures were performed using Altered Sites in vitro Mutagenesis System (Promega, Madison, Wis.).

i. Deletion Mutagenesis

Site-directed mutagenesis was performed using Altered Sites™ in vitro Mutagenesis System (Promega, Madison, Wis.). A unique SphI restriction site, (5′-GCATGC-3′), was generated within the SEC1 toxin disulfide loop coding region of the gene, sec⁺_{mndon}. This new site was used to linearize the mutated sec⁺_{mndon} gene by restriction endonuclease digestion. Following linearization by SphI endonuclease, bi-directional deletions using Bal 31 exonuclease (Boehringer Mannheim, Indianapolis, Ind.) were generated through timed digestions. The reaction mixture was composed of an equal volume of 2×Bal 31 enzyme buffer (24 mM CaCl₂, 24 mM MgCl₂, 0.4 mM NaCl, 40 mM Tris Base [pH 8.0], 2 mM EDTA) mixed with linearized SphI mutant sec⁺_{mndon} DNA. Digestion times were 0, 1, 2, 3, 4, 5, 10, 20, 30, 40, 50, 60, 90, and 120 minutes.

The six SEC1 deletion mutants are shown in FIG. 1. Four of the mutants: SEC1-4, SEC1-9, SEC1-12“G”, and SEC1-12“Y”, contained deletions within the disulfide loop structure. SEC1-4 and SEC1-9 had 4 and 9 deleted residues, respectively. SEC1-12“G” and SEC1-12“Y” both had twelve deleted residues. SEC1-12“G” and SEC1-12“Y” were so named because either residue 106G or 94Y, respectively, remained in the mutant loop region. SEC1-12“C” was a deletion mutant into which residues previously removed were replaced by non-native residues at the site of deletion. SEC1-12“C” was the result of the insertion of a single cysteine residue in a 13-residue deletion between 93C and 107G.

ii. Insertional Mutagenesis

A SEC1 mutant with an addition of six alanine residues (SEC1-12+6) was created using polymerase chain reaction (PCR). The sequence of the SEC1-12“G” loop mutant toxin gene (above) was used to design oligonucleotide primers for use in the PCR process. Two sets of primers were designed, each set containing a unique NotI (5′-GC^{↓}GGCCGC-3′) restriction site in either a 24-base 5′ or a 24-base 3′ extension. These primer sets were further designed so that the product of each would introduce one of two unique restriction sites found in either the N-terminal or the C-terminal region of SEC1. The N-terminal, 270 base pair (bp) product contained a BclI (5′-T^{↓}GATCA-3′) site and the C-terminal 318 bp product contained a NdeI (5′-CA^{↓}TATG-3′) site. Polymerase chain reaction amplification was performed using a Amplitron®II thermocycler (Barnstead/Thermolyne Dubuque, Iowa) with the following thermal profiles: 1 cycle, 97° C. for 5 min; 5 cycles, 95° C. for 1 min, 40° C. for 1 min and 72° C. for 1 min; 25 cycles, 95° C. for 1 min, 50° C. for 1 min and 72° C. for 1 min; 1 cycle 72° C. for 5: min.

C. Amplification

The mutant alleles were sub-cloned into pMIN164 (Iandolo, J. J. (1989) Annu. Rev. Microbiol. 43:375-402), a 8.6 Kb E. coli-S. aureus shuttle vector, and transferred to E. coli RR1 (Bolivar et al. (1977) Gene 2(2):95-113) for amplification.

D. Transformation (E. coli JM101)

Following mutagenesis, DNA fragments containing mutant sec⁺_{mndon} were ligated into pALTER™-1 and used to transform E. coli JM101. These strains were kept for subsequent characterization and stock culture production. Briefly, amplified products were agarose gel (1.0%) purified and subsequently double digested with the restriction endonucleases NotI and either BclI or NdeI for N-terminal and C-terminal products, respectively. Following digestion, fragments were ligated and agarose gel purified. The resultant 433 bp product was subsequently ligated into sec⁺_{mndon}, previously placed in the pALTER™-1 vector, at the BclI-NdeI restriction sites, and used to transform E. coli JM101.

E. Mutant Screening

Following mutagenesis, phagemid DNA was transformed into E. coli JM101, and ampicillin-resistant transformants were recovered from ampicillin-containing (125 μg/ml) Luria-Bertani media for further screening. Briefly, transformants showing ampicillin resistance were screened by Ouchterlony immunodiffusion (Ouchterlmy (1962) Prog. Allergy 6:30-54) using polyclonal rabbit antiserum against SEC1. Ampicillin-resistant transformants were transferred to and grown overnight in 1 ml broth cultures. Culture proteins were precipitated in four volumes of 100% ethanol at 4° C. for a minimum of 30 minutes. The precipitates were collected at the bottom of culture tubes by centrifugation for 10 min at 18,800-×g using a TJ-6 centrifuge (Beckman Instruments Inc., Palo Alto, Calif.). Pellets were dried in a vacuum chamber and resuspended in 30 μl of water. Ampicillin-resistant transformants were selected and evaluated for presence of the desired mutation.

F. DNA Sequencing

Dideoxynucleic acid sequencing methods (Sanger et al. (1977) Proc Natl Acad Sci USA. 74(12):5463-7) were used to confirm that the desired nucleotide mutations had been generated. VCS-M13 helper phage (Stratagene) was used to isolate phagemids carrying mutant sec⁺_{mndon} genes in the single stranded form. These single stranded phagemids served as templates for nucleotide sequencing. Sequencing reactions were performed using Sequenase Version 2.0, a commercially available kit (U.S. Biochemical Corp., Cleveland, Ohio). Radiolabled [³⁵S]-dATP DNA fragments were separated by electrophoresis in 7% polyacrylamide sequencing gels (1:29 N,N′-methylene-bis acrylamide to acrylamide w/v) and 8 M urea Electrophoresis was performed using an IBI sequencing apparatus international Biotechnologies Inc., New Haven, Conn.) and LKB model 2197 power supply using constant power of 60-70 watts. Autoradiography using Kodak X-OMAT™LS X-Ray film (Eastman Kodak Co., Rochester, N.Y.) was used to visualize DNA fragments in dried gels.

G. pMIN164 Plasmid

Plasmid pMIN164 was generated by ligation of staphylococcal plasmid pE194 to pBR328 (Hovde et al., (1990) Molecular and General Genetics, 220(2):329-333)

H. Expression of Mutant Sec⁺_{mndon}

The pMIN164 plasmid was transferred to S. aureus RN4220 (Couch et al. (1988) J Bacteriol. 170(7):2954-60) to facilitate purification of these mutant toxins and analysis of alteration in their biological activities. Briefly, pMIN164 was transferred to S. aureus RN4220, using standard protoplast procedures (Chang et al. (1979) Mol Gen Genet. 168(1): 111-5). The S. aureus RN4220 plasmid-containing transformants were maintained under erythromycin (50 μg/ml) selection.

### Example 2

### Purification of Mutant SEC1

The native and mutant SEC1 proteins were purified. Briefly, dialyzable beef heart media supplemented with 1% glucose buffer (330 mM glucose; 475 mM NaHCO₃; 680 mM NaCl; 137 mM Na₂HPO₄.H₂O; and 28 mM L-glutamine) (Schlievert et al. (1981) J Infect Dis. 143(4):509-16) was used for purification of native staphylococcal enterotoxin C1 (SEC1) and mutant staphylococcal enterotoxins (SE). Cultures were inoculated with 1 ml of an actively growing starter culture of S. aureus expressing the desired toxin and incubated overnight at 37° C. with vigorous shaking (200 rpm). Cultures of S. aureus RN4220 transformants carrying mutant sec⁺_{mndon}genes were grown under identical conditions, in media supplemented with erythromycin (50 μg/ml). Following overnight incubation, cultures were precipitated and left undisturbed for four days, in four volumes of 100% ethanol at 4° C. Extracellular protein precipitates were recovered by centrifugation (13,000×g) using a GSA rotor. After drying, the pellet was redissolved in water. The material that was insoluble in water was repelleted by centrifugation at 15,000 rpm (26,890×g) in a SS-34 rotor and discarded. The crude toxin solution was dialyzed overnight (MW cutoff 12,000-14,000) against pyrogen free water at 4° C. to remove salts and media components.

Purification of the remaining proteins was accomplished through successive preparative flat bed isoelectric focusing (IEF) using a Multiphor II (model 2197) electrophoresis system (LKB, Bromma, Sweden) (Winter et al. (1975) “Preparative flat-bed electrofocusing in a granulated gel with the LKB 2117 Multiphor”, LKB-Produkter-AB, Stockholm, Sweeden). The dialyzed crude toxin solution was initially pervaporated to a volume of between 50 and 100 ml. Following pervaporation 2.5 ml of 3.5-10 pI range ampholytes (KB), and an appropriate amount of crushed Sephadex (Sigma, St. Louis, Mo.) was added to the protein solution to create a thick slurry. This slurry was poured onto an endotoxin-free IEF plate having anode and cathode wicks placed at either end. The anode (+) and cathode (−) wicks were treated with 1 M H₃PO₄ and 1 M NaOH, respectively. After allowing the gel to dry to the appropriate state, it was electrophoresed overnight (16-20 hours) at 1000 volts, 20.0 amps, and 8.0 watts. The gel was subsequently sliced into fractions and each fraction was tested for the presence of toxin by Ouchterlony immunodiffusion with SEC1-specific rabbit anti-sera. Positive fractions were collected and subjected to a second IEF run as described above, using ampholytes of a narrower pI range. The ranges used were 7.0 through 9.0 or 6.0 through 8.0 depending on the toxins' expected pI. Fractions containing toxin, as detected by (SDS-PAGE), were pooled following removal of the Sephadex. Ampholytes were removed from the purified toxin by exhaustive dialysis (MW 12,000-14,000) for four days against pyrogen free water changed daily.

Proteins were transferred electrophoretically from SDS-PAGE slab gels to a nitrocellulose membrane (0.45 μm pore size) using the Mini-Protein II Trans Blot Apparatus (Bio-Rad). Transfer of proteins was completed in chilled western transfer buffer (1.52 M glycine, 250 mM Tris Base, 1.0% SDS, and 20% methanol) using a constant current of 150 mA for one hour. Prestained molecular weight standards were included to visually confirm protein transfer. Non-specific protein binding sites were blocked by incubating the membranes in 3% gelatin in TBS (0.02 M Tris base, 0.5 M NaCl, pH 7.5) at 37° C. for fifteen minutes. Nitrocellulose membranes were washed in TBS with 0.05% Tween 20 (Sigma, St. Louis, Mo.) (TBS-Tween) to remove gelatin and incubated overnight with the appropriate primary antibody (1:2500 dilution) in TBS-Tween. The filter was then subjected to three washes in TBS-Tween to remove any unbound primary antibody. Subsequent to the washes, the membrane was incubated with an alkaline phosphatase-conjugated species-specific anti-immunoglobulin (1:5000 dilution) in TBS-Tween for two hours at room temperature. The membrane was again washed and processed in a indoxyl phosphate-nitroblue tetrazolium system (18 ml sodium barbital buffer [pH 9.6], 2.0 ml 0.1% nitroblue tetrozolium, 40 μl 2 M MgCl2, and 2 mg 5-bromo, 4-chloro-indoxylphosphate in 0.4 ml dimethylformamide) (Blake et al. (1984) Anal Biochem. 136(1):175-9) to visualize antigen/antibody complexes remaining on the membrane. The reaction was stopped with several washes of distilled water.

Yields of SEC1 mutant toxins were found to be from 10 to 60% lower than yields obtained from wild type SEC1 (5 mg/L culture) when purified from equal volumes of culture grown under identical conditions. (data not shown)

### Example 3

### Molecular Weights of Mutant SEC1

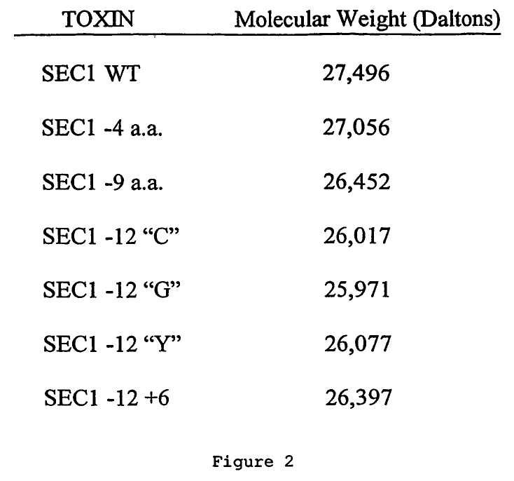

The molecular weights of the mutant SEC1 toxins were compared (FIG. 2) using SDS-polyacrylamide gel electrophoresis (SDS-PAGE). Briefly, SDS-PAGE was preformed using a Mini-Protein II slab gel apparatus (Bio-Rad, Richmond, Calif.). The resolving gel was 12.5% acrylamide (1:36.5 N,N′-methylene-bis acrylamide to acrylamide) and the stacking gel was 4.5% acrylamide. Samples were prepared by mixing with 5×sample buffer (50 mM Tris-Cl pH 6.8, 100 mM 2-mercaptoethanol, 2% SDS, 0.1% bromophenol blue and 10% glycerol) and heating at 100° C. for five minutes. Electrophoresis was conducted in a Tris-glycine buffer system (25 mM Tris, 250 mM glycine, 0.1% SDS) at 120 volts until the dye front migrated off the gel. The gels were either stained with Coomassie Brilliant Blue R-250 for two hours, or transferred to nitrocellulose (see below). After staining, proteins were visualized by destaining in 20% acetic acid, 20% methanol until the background was colorless. SDS-PAGE prestained molecular weight standards W 12,400-95,500) (Diversified Biotech, Boston, Mass.) were used to determine position of toxin bands.

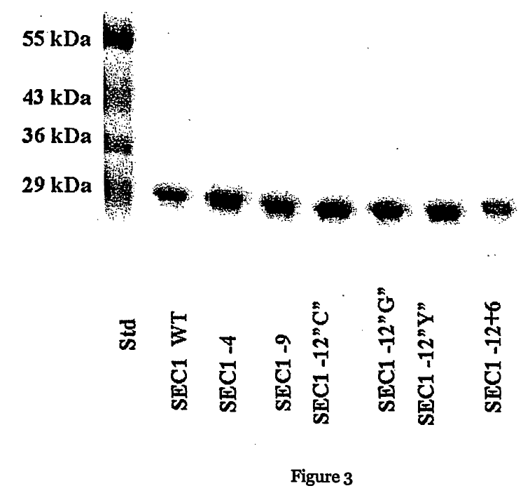

The molecular weights of the mutant toxins closely approximated that of the native SEC1 protein (FIG. 3).

### Example 4

### Relatedness of Native and Mutant Enterotoxins

The relatedness of the native an mutant enterotoxins was determined by immunodiffusion following the method of Ouchterlony (Ouchterlony, O. (1962) Prog Allergy. 6:30-54). Briefly, Hyperimmune polyclonal antiserum was used to immuno-percipitate protein in an agarose matrix. The gel matrix was prepared by applying 5 ml of molten agarose (0.75%) in phosphate buffered saline (PBS) (pH 7.2-7.4) to a microscope slide. Test wells were punched in the solidified agarose using an immunodiffusion template (LKB). Antiserum was placed in the center well with antigen test samples around it so that antigen and antibody could diffuse towards each other. The slides were incubated for four hours at 37° C. or overnight at room temperature (22° C.). Lines of precipitation were visualized under a fluorescent light (Hyperion viewer with a magnifier; Hyperion, Inc., Miami, Fla.). Therefore, the proteins were indistinguishable antigenically.

### Example 5

### Proteolytic Liability

The biological effects of SE toxins appear to be related to the structural stability of the toxin. Alterations in the native structure of the toxin have been shown to affect protein stability and reduce the ability to induce the biological activities associated with these toxins (Grossman et al. (1990) J Exp Med. 172(6):1831-41; Grossman et al. (1991) J Immunol. 147(10):3274-81; Hovde et al. (1994) Mol Microbiol. 13(5):897-909; Kappler et al. (1992) J Exp Med. 175(2):387-96.). Trypsin, pepsin, and gastric fluid liability assays were employed to determine any significant changes in stability of the six SEC1 mutants used in this study.

A. Trypsin

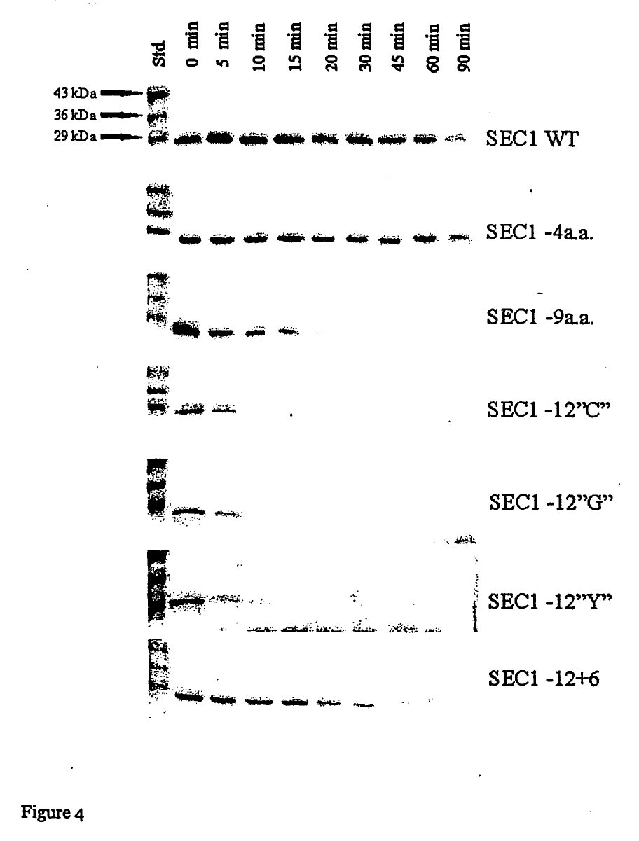

Native SEC1 has 34 potential tryptic cleavage sites, 14 located in domain 1, three of which are located in the disulfide loop; K98, K103, and K108. Despite this, peptide bonds at lysine residues 59 and 103 of the native toxin have been shown to be highly susceptible to cleavage by trypsin (Hovde et al. (1994) Mol Microbiol. 13(5):897-909). The bond at residue 103, located within the disulfide loop of SEC1, has been deleted in all six of the SEC1 mutants (FIG. 1). With removal of one of the two-trypsin sensitive residues, the relative rates of tryptic digestion for each of the loop deletion mutants were compared to that of SEC1 and visualized using SDS-PAGE (FIG. 4).

Trypsin type XI (Sigma, St. Louis, Mo.) was used to compare degradation patterns of native SEC1 and SEC1 mutant toxins. Fifteen μl of purified native or mutant toxin (1.0 μg/μl) was mixed with trypsin to a final concentration of 80 μg/ml of trypsin, and incubated at 37° C. in a timed digestion. Digestion time points were 0, 5, 10, 15, 30, 45, 60, and 90 minutes. Following incubation, the trypsin digestions were terminated by heating at 100° C. for 5 minutes in SDS-PAGE sample buffer (50 mM Tris-Cl pH 6.8, 100 mM 2-mercaptoethanol, 2% SDS, 0.1% bromophenol blue and 10% glycerol). Following SDS-PAGE, gels were stained with Coomassie Brilliant Blue R-250 for 2 hours and destained.

The rate at which trypsin degraded each mutant toxin was related to the number of residues deleted from the loop. The SEC1-4 mutant toxin, the most stable of the SEC1 mutants, had a tryptic digestion rate that was indistinguishable from SEC1 wild type toxin; indicating no apparent structural instability of this mutant relative to the native SEC1. The remaining five deletion mutants had increased susceptibility to tryptic digestion. This increase in susceptibility to tryptic digestion suggests that conformational alterations had occurred in these toxins. These alterations most likely resulted in increased accessibility of trypsin to alternative tryptic cleavage sites. Interestingly, three of the SEC1-12 loop mutants, SEC1-12“Y”, SEC1-12“G”, and SEC1-12“C” showed differences in digestive rates. The SEC1-12“Y” mutant was the most resistant followed by SEC1-12“G” and SEC1-12“C”. The SEC1-12+6 mutant, containing four native and six non-native residues, showed a decrease to proteolytic cleavage relative to that of mutants having larger net deletions. This suggests that the SEC1-12+6 toxin variant is more stable and that this stability is not completely dependent on the presence of the normally resident native amino acids.

B. Pepsin

SEC1 native and mutant toxins were treated with Pepsin (Sigma, St. Louis, Mo.) to compare relative degradation rates. Ten μl of purified native or mutant toxin (1.0 μg/μl) was mixed with 15 μl pepsin (500 μg/ml in 100 mM NaOAc pH4.5) and digested at 37° C. in a total volume of 100 μl for timed digestion. Time points were 5, 10, 15, 20, 30, 45 and 60 minutes. Analysis was as described for trypsin-tested samples.

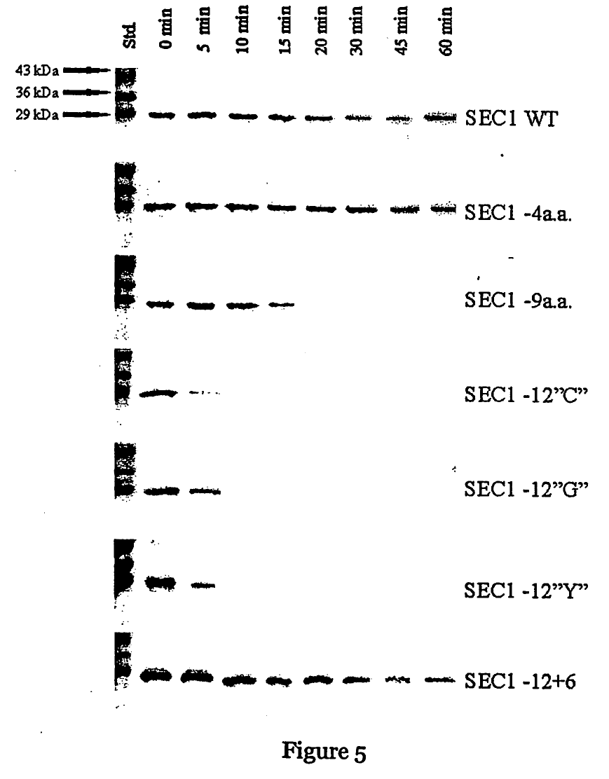

Pepsin degradation of the SEC1 loop mutants showed nearly identical digestion rates (FIG. 5) to those observed using trypsin. Again, as the deletions became larger the rate at which the proteins were degraded was found to increase.

C. Gastric Fluid

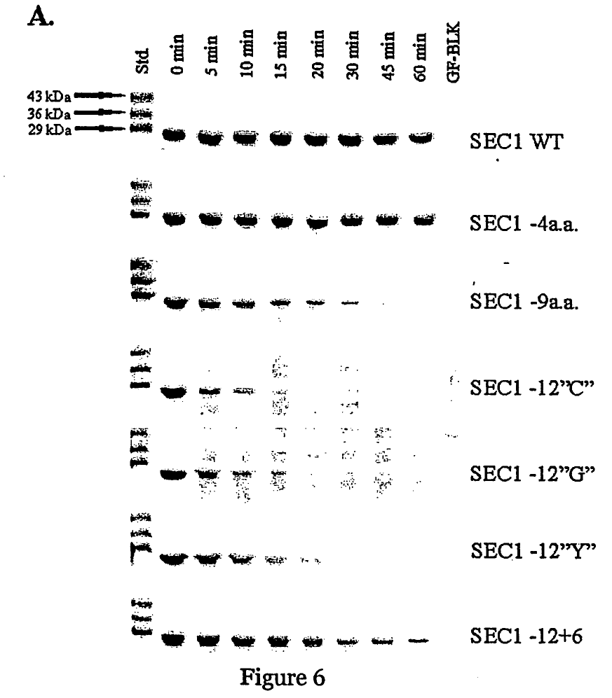

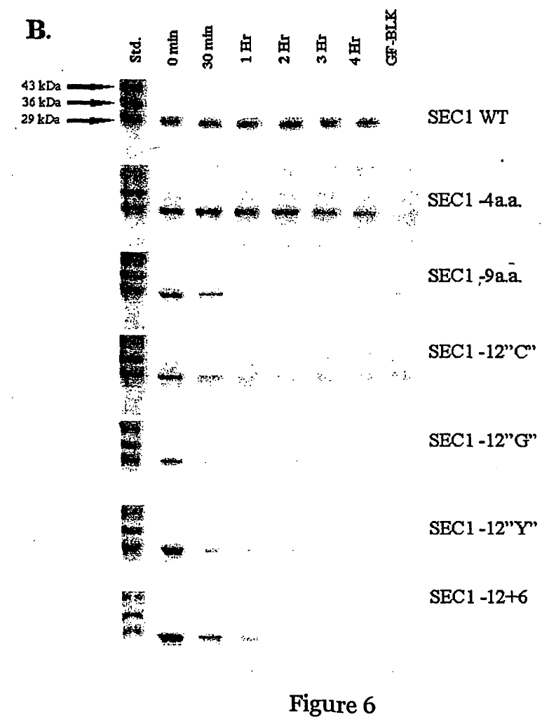

Gastric fluid stability was assessed to determine the stability of the toxin in the gastrointestinal tract. Two time course experiments were performed, a one hour digest (FIG. 6A), and a four hour digest (FIG. 6B). Gastric fluid was obtained by saline lavage through a nasogastric tube from the stomach of Macaca nemestrina monkeys. Native and mutant toxin was incubated at 37° C. in dilute gastric lavage fluid (1:2 in sterile physiological saline) for a timed digestion. Time points were 0, 5, 10, 15, 30, 45, 60, and 90 minutes. After inactivation of enzymatic activity, SDS-PAGE analysis was used to visualize toxin degradation.

As was observed in the digestive patterns for trypsin and pepsin, both components of gastric fluid, SEC1-4 was indistinguishable from the native SEC1 toxin. The degree of increasing susceptibility to degradation of the remaining mutant toxins was directly related to the size of the loop deletion. As was seen previously with the SEC1-12 mutants, SEC1-12“Y” was found to be the most resistant of the three toxins followed by SEC1-12“G” and SEC1-12“C”.

D. Conclusion Re: Proteolysis

All three proteolytic assays demonstrated that the SEC-12 mutant toxins were the most susceptible to proteolytic digestion and, most likely the least stable of the SEC1 mutants. Reduction in resistance to proteolytic degradation, presumably due to alterations in loop size, were most pronounced in the SEC1-12“Y”, SEC1-12“G”, and SEC1-12“C” mutants. This suggests that the largest degree of alteration to the structural conformation had occurred within the toxins wherein the disulfide loop structure has been almost completely removed.

Generally, as the size of the loop deletion became larger, the toxins became more susceptible to proteolytic digestion. Partial restoration of the native loop size by the insertion of six non-native alanine residues into an SEC1 mutant containing a twelve amino acid deletion increased resistance to proteolytic degradation, similar to other SEC1 mutants having an equivalent deletion size.

A decrease in stability, resulting in a more rapid proteolytic degradation rate then that of the native SEC1, is also evidence supporting that a change to the overall structural conformation of the toxin had occurred.

### Example 6

### Biological Activity

A. Emesis Assay.

The emetic ability of the SEs is a unique biological activity that separates this group from other PTs. The ability of the six SEC1 loop mutants to induce emesis was assessed using a monkey model. Two experimental procedures were used. The first was a modification of the standard monkey feeding assay for staphylococcal enterotoxin (Bergdoll, M. S. (1988). Methods Enzymol. 165:324-33). The second was a syringe feeding assay. In each experimental method sterile physiological saline was administered to serve as a negative control.

i. Standard Feeding Assay

Animals involved in the modified standard feeding assay were manually restrained while toxin, resolublized in sterile physiological saline, was administered through a nasogastric tube (Infant feeding tube; Becton Dickinson, Rutherford, N.J.). After inoculation of toxin and removal of the nasogastric tube, animals were returned to their cages and observed for a minimum of 12 hours for an emetic response. Toxins were administered at a concentration range from 1 μg/kg of toxin to body weight up to 250 μg/kg of toxin to body weight. Initial screening for retention of emetic activity was performed at a dose of 10 μg/kg, which is approximately 100 times the minimal emetic dose for SEC1 (Hovde et al. (1994) Mol. Microbiol. 13(5):897-909). Toxins showing emesis at the initial concentration of 10 μg/kg were readministered at a log fold decrease, 1 μg/kg. Non-emetic toxins were tested for residual emetic activity at a high dose of 250 μg/kg.

ii. Syringe Feeding Assay

Animals involved in the syringe feeding assay procedures were administered dissolved SEC1 native or mutant toxin in a commercially available fruit punch slush flavor (Lyons-Magnus, Clovis, Calif.) using a syringe. Following syringe feeding, the monkeys were observed again for a minimum of 12 hours for an emetic response. The initial toxin dose of 10 μg/kg was shown in this study to be 10 times the minimal emetic dose for SEC1. Toxins showing emesis at the initial dose were re-administered at a log fold decrease. Non-emetic toxins were tested for residual emetic activity at a log fold increase from the initial dose.

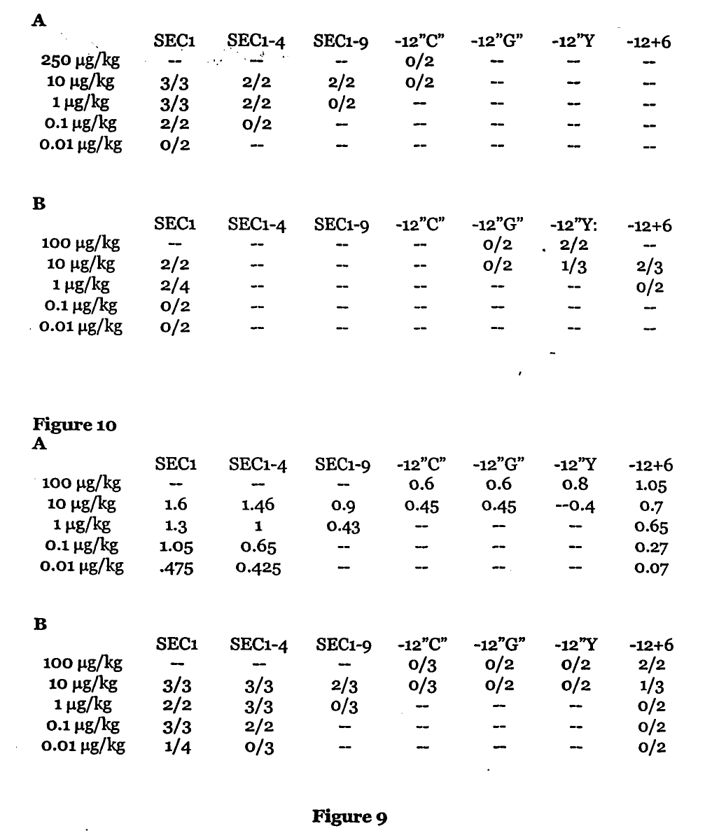

The mutants SEC1-12“G” and SEC1-12“C”, when administered at doses up to 100 μg/kg and 250 μg/kg respectively, showed no emetic capability whatsoever. All other SEC1 loop mutants did exhibit some degree of emetic capability though potency varied between mutants (FIG. 9). The SEC1-4 mutant possessed an emetic ability very similar to that of SEC1, presumably due to the large portion of loop structure still being present. In all mutants, as the loop deletion became larger, minimal emetic dose also became larger. This relation of loop size to emetic ability can possibly be related to toxin stability in the gastrointestinal tract. The more susceptible to degradation each mutant was, as was determined in the proteolytic analysis, the less able it was to induce emesis. In agreement with Hovde et al. (Hovde et al. (1994) Mol Microbiol. 13(5):897-909), the disulfide bond appeared to be involved in stabilization of a protein conformation required for emetic activity. This was demonstrated with SEC1-12“C”, a loop mutant lacking the disulfide linkage and having no emetic activity. The SEC1-12“Y” and SEC1-12“G” mutants, both with a disulfide linkage showed differing results in that the SEC1-12“G” lacked emesis and SEC1-12“Y” retained some degree of biological activity. The inability of SEC1-12“G” to induce emesis, unlike SEC1-12“Y”, might be correlated to the increased stability suggested by the proteolytic studies comparing the two toxins. The SEC1-12“G” mutant was demonstrated to be equally as unstable as the SEC1-12“C” mutant, which was also unable to induce emesis. These results support the hypothesis that emetic activity is not directly related to the loop structure but instead to another region on the protein stabilized by the loop region of the toxin.

B. Mitogenicity.

The mitogenic capacity of mutant toxins was compared to that of native toxin using human peripheral blood mononuclear cells (PMBC) in a standard 4-day assay (Poindexter et al. (1987) J Infect Dis. 156(l):122-9). Collection of PMBC started with whole blood collected from human volunteers by venipuncture into Vacutainer tubes. Once taken, clotting of whole blood was prevented by adding Heparin (Sigma, St. Louis, Mo.) (150 U/25 ml). The eparinized blood was layered on a Ficoll-Paque (6:4 v:v, blood:Ficoll-Paque) gradient and centrifuged for 15 min at 500×g using a TH-4 rotor for separation of the mononuclear cells. PMBC were recovered from the middle white layer and washed with Hanks buffered saline solution. After washing, cells were collected by centrifugation using a TH-4 rotor at 250×g for 10 min, and resuspended in complete RPMI media containing 2% fetal bovine serum FBS), 2 mM glutamine, 200 U/ml sodium penicillin G, and 200 μg/ml streptomycin sulfate. Cell density was determined and adjusted to a concentration of 1×10⁶ cells/ml. Two hundred μl aliquots of this cell suspension were placed in the wells of a 96 well tissue culture plate (Costar, Cambridge, Mass.).

Solutions of native and mutant toxin were added, in triplicate wells, to the cell suspensions in the amounts of 1.0 μg, 0.1 μg, 0.01 μg, 1.0 pg, 0.1 pg, 0.01 pg, 1.0 ng and 0.01 ng. Concanavalin-A added to cultures in the amounts of 1.0 μg, and 0.1 μg served as positive controls while RPMI media alone served as a negative control. Toxin-treated cells were incubated at 37° C./6% CO₂ for 72 hours. Following incubation, 1 μCi [³H]-thymidine (New Research Products, Boston, Mass.) (25 μl in a complete RPMI medium) was added to each well and allowed to incubate under the same conditions for an additional 18-24 hours. After incubation, radiolabled cells were harvested onto glass fiber filters (Skatron, Sterling, Va.) using a semi-automatic cell harvester (Skatron). Measuring incorporation of [3H]-thymidine into cellular DNA quantitated lymphocyte proliferation. Incorporation of [³H]-thymidine into the DNA of replicating cells was quantified on a liquid scintillation counter (TRI-CARB 1500 Liquid Scintillation Counter, Packard, Rockville, Md.).

T-cell proliferation has been thought to play a role in the symptoms observed in SE disease due to the large associated cytokine release (Bohach et al. (1996) “The staphylococcal and streptococcal pyrogenic toxin family.”, In B. R. Singh and A. T. Tu (ed.), Natural Toxins II. Plenum Press, New York., p. 131-154.). Ability of SEC1 and SEC1 mutants to stimulate T-cells was quantitated using enriched human peripheral blood mononuclear cells collected from volunteers.

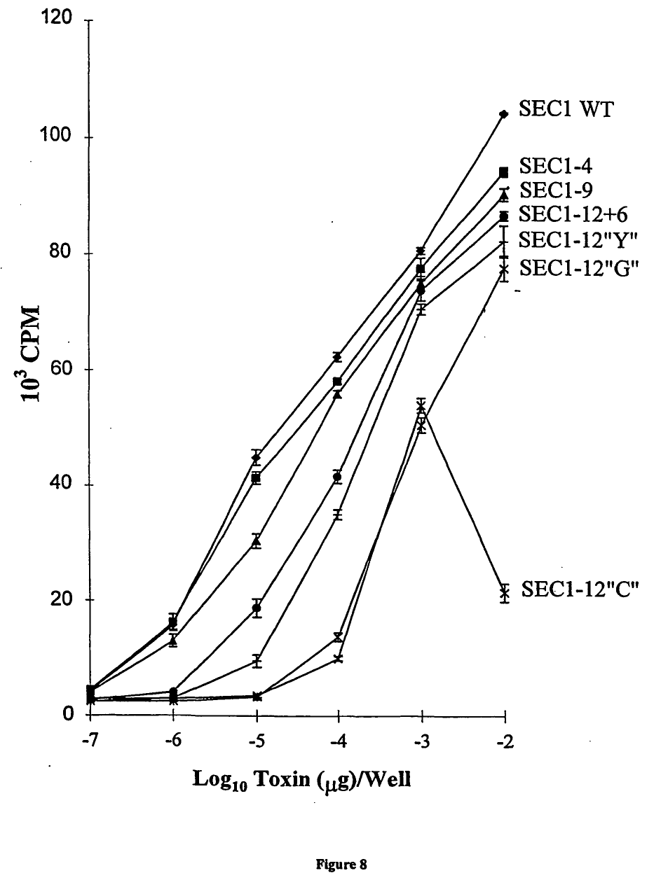

Although all of the mutants showed some level of mitogenic activity (FIG. 8), there was a wide range in their stimulatory effectiveness. Mitogenic response of wild type SEC1 showed a nearly linear increase of T-cell proliferation at a concentration range of 10⁻⁷ to 10⁻¹ μg toxin/well. Similar linear proliferative responses were seen in the SEC1-4 and SEC1-9 mutants. SEC1-4 had an almost identical stimulatory capacity to that of SEC1. Though not as potent a stimulator as either SEC1 or SEC1-4, SEC1-9 was shown to have the same proliferative dose range. Lower in dose potency were the SEC1-12 loop mutants.

The SEC1-12“Y” and SEC1-12+6 mutants produced a proliferative response in a narrower dose range, 10⁻⁶ to 10⁻² μg toxin/well then that of the native SEC1. The SEC1-12“G” mutant was found to induce its greatest proliferative response at a protein concentration 10⁻² μg toxin/well, very much like the SEC1-12“Y” and SEC1-12+6 toxins. At doses lower than 10⁻² μg toxin/well its proliferative ability diminished rapidly. Very similar to SEC1-12“G”, at doses up to 10⁻³ μg toxin/well, SEC1-12“C” lost its T-cell proliferative ability above that concentration and showed a rapid decrease thereafter. When compared to stimulation by wild type SEC1, equivalent stimulation observed from the SEC1-12 mutants required a 10 to 100 fold increase in toxin dose.

These results demonstrate that the loop structure has a role in the mitogenic ability of the SEs. Mitogenic potency of each mutant decreased as the size of the loop decreased. Additionally, in agreement with previous studies showing that a disruption of the disulfide bond reduced mitogenic capability (Grossman et al (1990) J Exp Med. 172(6):1831-41; Grossman et al. (1991) J Immunol. 147(10):3274-81; Hovde et al. (1994) Mol Microbiol. 13(5):897-909; Kappler et al. (1992) J Exp Med. 175(2):387-96.), there seemed to be a requirement for the presence of the disulfide bond, as noted by the decreased mitogenic capability of the SEC1-12“C” mutant. The mitogenic capability seen with SEC1-12+6 supports the hypothesis that it is not wholly residues within the loop that are required for mitogenicity but rather the size of loop structure itself.

C. Pyrogenicity and Enhancement of Lethal Endotoxic Shock.

The ability to induce a fever and enhance shock susceptibility is a well-characterized biological activity of the PTs (Bohach et al. (1990) Crit Rev Microbiol. 17(4):251-72; Bohach et al. (1996) “The staphylococcal and streptococcal pyrogenic toxin family.”, In B. R. Singh and A. T. Tu (ed.), Natural Toxins II. Plenum Press, New York., p. 131-154). To examine the role of the disulfide loop in biological activity, six SEC1 mutants were tested for their ability to induce a pyrogenic response and enhance lethal susceptibility to endotoxic shock in a rabbit model (Kim et al. (1970) J Exp Med. 131(3):611-22).

The ability of SEC1 and the SEC1 mutant toxins to induce a fever response and enhance susceptibility of lethal endotoxic shock was determined in vivo using the rabbit model described by Kim and Watson (Kim et al. (1970) J Exp Med. 131(3):611-22). Adult New Zealand White rabbits used in the assay were initially preconditioned for one hour in a test rack and had their baseline body temperature recorded. The animals subsequently received an intravenous injection containing 10-μg/kg body weight of test toxin suspended in sterile physiological saline. Sterile saline and purified SEC1 were used as negative and positive controls, respectively. Following toxin injection, rabbit body temperature was monitored rectally every hour for four hours using the YSI Model 42SC Tele-Thermometer with reusable probes (Yellow Springs Instrument Co., Yellow Springs, Ohio). Four hours after initial treatment, an intravenous injection of lipopolysaccharide UPS) from Salmonella typhimurium (Difco Laboratories, Detroit, Mich.) was administered at a concentration of 10 μg/kg in sterile saline. Animals were observed for signs of shock and mortality for 48 hours after receiving LPS injection.

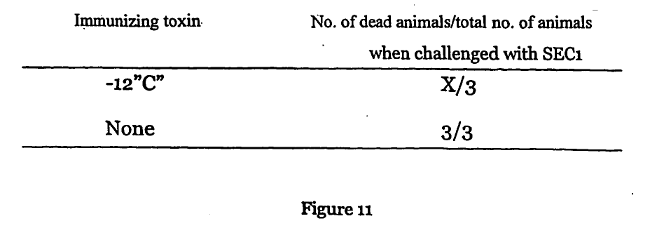

Wild type SEC1 induces a pyrogenic response (FIG. 10A) and results in an enhanced susceptibility to lethal endotoxic shock (FIG. 10B). The ability of the SEC1 mutants to induce fever and enhance susceptibility to lethal endotoxic shock was found to decrease as the size of the loop decreased. Mutants SEC1-4, SEC1-9 and SEC1-12+6 exhibited both fever and created an increased susceptibility to lethal endotoxic shock in this animal model. However, while these mutants all showed biological activity, only the SEC1-4 mutant induced biological responses at doses similar to those of the native SEC1 toxin. The ability of SEC1-9 and SEC1-12+6 loop mutants to induce fever and increase susceptibility to lethal endotoxic shock was greatly reduced. The ability to increase susceptibility to lethal endotoxic shock by SEC1-12“Y”, SEC1-12“G”, and SEC1-12“C” was absent, even in animals having a 10 fold increase above the initial toxin dose (10 μg/kg body weight). While able to induce a fever in test animals, a 1,000-fold dose increase was required to elicit a response similar to that of SEC1.

D. Conclusion Re: Biological Activity

Reductions in the ability to induce biological activity of the SEC1 mutant toxins were most pronounced in the SEC1-12“Y”, SEC1-12“-G”, and SEC1-12“C” mutants. Generally, as the size of the loop deletion became larger, the toxins became less able to induce SE biological activities. Partial restoration of the native loop size by the insertion of six non-native alanine residues into an SEC1 mutant containing a twelve amino acid deletion increased the ability to induce biological activities, similar to other SEC1 mutants having an equivalent deletion size. These results clearly demonstrate that the disulfide loop structure is involved in induction of biological activity, perhaps by reducing the susceptibility of the toxin to proteolytic degradation, thus allowing the toxin to persist longer within the host.

### Example 7

### Disulfide Bond Determination

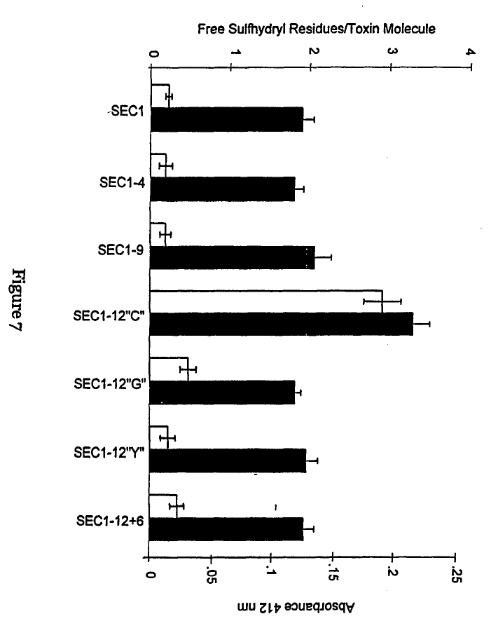

The presence of the disulfide bond in SEs has been previously reported to be related to the biological activities of the toxin, as well as its structural stability (Grossman et al. (1990) J Exp Med. 172(6):1831-41; Grossman et al. (1991) J Immunol. 147(10):3274-81; Hovde et al. (1994) Mol Microbiol. 13(5):897-909; Kappler et al. (1992) J Exp Med. 175(2):387-96). To determine whether the results obtained using SEC1 mutants (in the Examples above) were due to the specific deletions and not the absence of the disulfide bond, each toxin was assayed for the presence of free sulfhydryls. 5,5′-Dithio-bis(2-Nitrobenzoic Acid) (DTNB), a compound that reacts with free sulfhydryl side chains, was used to spectrophotometrically determine if free sulfhydryls were present in native SEC1 and the six SEC1 mutant toxins (FIG. 7).

Disulfide bond determination was accomplished by measuring the presence of unbound toxin sulfhydryls in solution, using a modification of a previously described procedure (Robyt et al. (1971) Arch Biochem Biophys. 147(1):262-9), under both reducing and non-reducing conditions. The reaction mixture was 5×10⁻⁶M of purified SEC1 or SEC1 loop mutant toxin, 1 mM 5,5′-Dithio-bis(2-Nitrobenzoic Acid) (DTNB)(Sigma; St. Louis, Mo.) and 1M phosphate buffer (pH 8.1) in a total volume of 1 ml for non-reducing reactions. Reducing reactions contained 10⁻² mM 2-mercaptoethanol. Following addition of DTNB, the sample was incubated for 30 minutes at room temperature (22° C.). Immediately following the incubation, toxins' free sulfhydryl content was determined spectrophotometically at 412 nm. Absorbance measurements were converted to number of sulfhydryl residues per toxin molecule using the molar extinction coefficient 13,600/cm (Robyt et al. (1971) Arch Biochem Biophys. 147(1):262-9).

Reactivity of 5,5′-Dithio-bis (2-Nitrobenzoic Acid) (DTNB) with free SH groups showed that all of the mutant toxins, with the exception of SEC1-12“C”, had maintained their native disulfide bond. The SEC1-12“C” toxin, with a third cysteine, reacted with DTNB at a level approximately 50% higher then the level of reduced SEC1 in both the reduced and non-reduced assays. Of note was a 10% reduction in reactivity seen between the SEC1-12“C” mutant under non-reducing conditions. This reduction suggests that there may be a low level of inter-molecular bonding between molecules of the −12“C” mutant that might account for some of the results this mutant demonstrated, particularly the rapid loss of mitogenic potency at concentrations above 5×10⁻³ μg/ml. Though the level of measured reactivity between DTNB and free SH in the SEC1-12“Y”, SEC1-12“G” and SEC1-12+6 were elevated above that of native SEC1 they were still at most, in the case of the SEC1-12“G” mutant, <30% of the measured values for reduced toxins indicating that a bond had formed (FIG. 7).

### Example 8

### Rabbit Protection Assay

For protection assays, the SEC1-12“C” construct was chosen as the mutant most likely to induce a protective immune response against the biologically active SEC1 in a rabbit model while producing the least toxic effects when administered. Adult New Zealand White rabbits were immunized with 25 μg of purified toxin in a 250-μl volume of sterile physiological saline. The toxin preparation was suspended in an equal volume of Freund's adjuvant (Sigma) and mixed thoroughly before injection as described by Schlievert et al. (1977) Infect Immun. 16(2):673-9. Immunizations were continued until serum antibodies specific to the SEC1-12“C” toxin were detected by Ouchterloney immunodiffusion. Seven days after the final booster, the rabbits were challenged with an intravenous injection of native SEC1 (5 μg/kg) in sterile saline. Following toxin injection, rabbit body temperature was monitored rectally every hour for four hours as described above. Four hours after initial treatment, an intravenous injection of endotoxin (10 μg/kg in sterile saline) from Salmonella typhimurium Difco Laboratories, Detroit, Mich.) was administered. Animals were observed for signs of shock and mortality for 48 hours after receiving endotoxin injection. At least some of the inoculated rabbits displayed protection.

II. Plants

Experiments were undertaken to express genetically modified SE's, with reduced toxic properties but intact antigenic determinants, in plant tissue using a recombinant tobacco mosaic virus-based system (rTMV-30B).

### Example 9

### Preparation of the rTMV-30B Expression System

An SEC1 mutant, SEC1-12C (Callantine et al. (2000) The role of the disulfide loop in the biological activity of Staphylococcal enterotoxin C1. in press), and a novel SE chimera containing the n-terminal half of SEC1-12C and the c-terminal half of SEA (SEC1-12C/SEA) were cloned into a rTMV-30B expression system. The Callantine SE type C1 mutant was used because it has previously been shown to contain the antigenic determinants necessary to induce immunological protection in rabbit model and attenuated biological properties associated with the native toxin.

DNA manipulations were performed according to routine methods (Sambrook et al., (1989) Molecular Cloning: A Laboratory Manual, 2nd 3d., Cold Spring Harbor Laboratory Press; Cold Spring Harbor, N.Y.). All DNA modifying enzymes and Concert™ plasmid DNA purification kits were purchased form GibCo-BRL, unless otherwise noted.

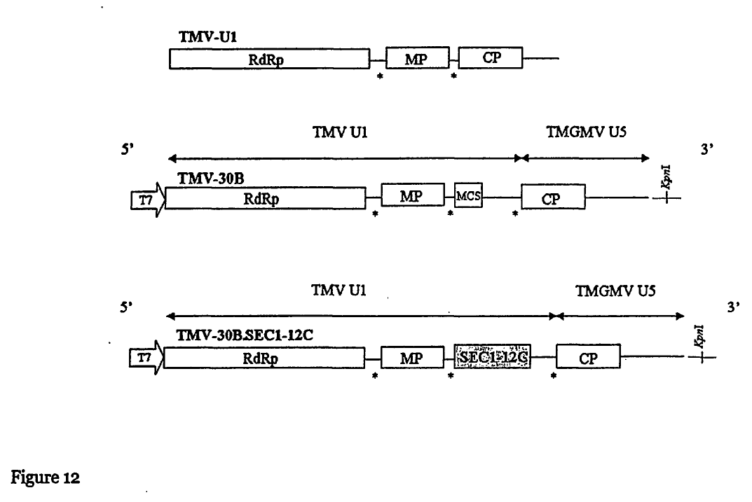

Briefly, an infectious TMV cDNA clone (p30B.TMV) was used. These cDNA clones are extensively modified derivatives of the TMV U1 strain (FIG. 12). The native coat protein (CP) open reading frame (ORF) has been modified to serve as a cloning site for the insertion of a foreign gene transcribed by the native CP subgenomic mRNA promoter. A heterologous subgenomic promoter, CP ORF, and nontranslated 3′ sequence was adapted from tobacco mild green mosaic virus (TMGMV) strain U5.

The mature SEC1-12C gene was obtained by PCR amplification from a pALTER™-1 clone (Beachy et al (1996) Ann N Y Acad Sci. 792:43-9) and subcloned into pET24d (Novagen, Madison, Wis.) using the NcoI and NotI sites provided in the multiple cloning site. The primer SEC1-12C/N was used to introduce the start codon, NcoI restriction site and a plant Kozak consensus sequence (Bohach et al (1997) Exotoxins, p. 83-111. In K. B. Crossley and G. L. Archer (ed.), The Staphylococci in Human Disease. Churchhill Livingstone, N.Y.). The primer SEC1-12C/C was used to remove the native terminator and introduce a NotI restriction site, keeping the reading frame necessary to incorporate the vector histidine tag and terminator.

| | |
|---|---|
| SEC1-12C/N primer: | 5′ CCGCCATGGCAAGCTTAA CAATGGCAGA |
| | GAGCCAA 3′ |
| SEC1-12C/C primer: | 5′ CCTATCAGCGGCCGCG GATCCATTCTTT |
| | GTTGT 3′ |

The SE chimera containing the N-terminus of SEC1-12C and the C-terminus of SEA was constructed using PCR based mutagenesis. The SEC1-12C gene was amplified using the primers SEC1-12C/N (described above) and 3′SEC-CLA, which introduced a unique ClaI restriction site located at nucleotide 445 of the wildtype (wt.) sec1 gene.

| | |
|---|---|
| 3′SEC-CLA primer: | 5′ CCCATTATCAAATCGATT TCCTTCATGT |
| | TTTG 3′ |

The wt. sea gene was obtained from S. aureus strain FRI913 (Bohach et al. (1990) Crit Rev Microbiol. 17(4):251-72) by PCR amplification. The primer 5′SEA-CLA utilized the ClaI restriction site at nucleotide 424 (Arakawa et al (1997) Transgenic Res. 6(6):403-13). The primer 3′SEA removed the native terminator and introduced a NotI restriction site for utilization of the vector histidine tag and terminator.

| | |
|---|---|
| 5′SEA-CLA primer: | 5′ CATGATAATAATCGATTGACCGAAGAGAA |
| | AAAAGTGCCG 3′ |
| 3′SEA primer: | 5′ TTTCTCGAGTGCGGCCGCACTTGTATATA |
| | AATATATATCAATATGC 3′ |

The SEC1-12C NcoI/ClaI fragment and the ClaI/NotI fragment of SEA were co-ligated into pET24d using the NcoI and NotI sites.

The resulting plasmids, pET24d.SEC1-12C and pET24d.SEC1-12C/SEA, were used as the template for PCR amplification and cloning into the p30B.TMV plasmid. The primers 5′ 30B-PAC and 3′ 30B-PME incorporated PacI and PmeI sites, respectively, while also utilizing the plant Kozak sequence and pET24d histidine tag.

| | |
|---|---|
| 5′ 30B-PAC primer: | 5′ CCGCGGTTAATTAAGCTTAACAATGGC |
| | 3′ |
| 3′ 30B-PME primer: | 5′ CATGCGTTTAAACTCTAGATTATCAGTGG |
| | TG 3′ |

Construction of the plasmid p30B.SEC1-12C was facilitated by blunt-ended cloning into the PmeI restriction site, while the plasmid p30B.SEC1-12C/SEA was constructed using both the PacI and PmeI sites. Restriction digests of purified plasmids ensured the proper orientation-of the SE genes in the p30B polylinker. To verify the fidelity of all constructs, DNA sequencing was performed commercially by Amplicon Express (Pullman, Wash.).

### Example 10

### In vitro Infection of Nicotiana benthamiana

Recombinant vrial infections with rTMV-30B.SEC1-12C and rTMV-30B.SEC1-12C/SEA were established in Nicotiana benthamiana plants using in vitro derived infectious rTMV-RNA transcripts. A p30B-derivative containing a green fluorescent protein (GFP) reporter gene (p30B.GFP) was used as a positive control (Shivprasad et al. (1999) Virology. 255(2):312-23).

The synthesis of in vitro transcripts was performed as a modification of the procedure described by Lewandowski and Dawson (Lewandowski et al. (1998) Virology. 251(2):427-37). Briefly, for each 25 μl reaction, 2.5 μg of KpnI linearized p30B.SEC1-12C DNA was added as template. The T7 transcription reaction consisted of 1×T7 RNA polymerase buffer (40 mM Tris-HCl, 6 MM MgCl₂, 2 mM spermidine, 10 mM dithiothreital, pH 7.9) (New England Biolabs, Beverly, Mass.); 10 mM dithiothreital (Gibb BRL); 25 mM each ATP, CTP, and UTP (Amersham-Pharmacia, Piscataway, N.J.); 0.25 mM GTP (Amersham-Pharmacia); 100 mM MgCl₂ (Gibb BRL); 6.25 mM cap analogue (Diguanosine Triphosphate) (Amersham-Pharmacia); and 20 U of recombinant RNasin (Promega, Madison, Wis.). After a two minute incubation at 37° C., 50 U of T7 RNA-polymerase (New England Biolabs) were added and the reaction allowed to continue for an additional 15 minutes. The GTP concentration was then adjusted to 27 mM and the reaction was incubated an additional 75 minutes at 37° C. Immediately following the transcription reaction, the infectious RNA transcripts were placed on ice and 25 μl of diethylpyrocarbonate (DEPC) treated water was added. The samples were gently mixed in an equal volume of ice cold FES buffer (0.5 M glycine, 0.3 M K₂HPO₄, 1% sodium pyrophosphate, 1% macaloid, 1% celite (pH 9.0)) before mechanical inoculation of carborundum-dusted Nicotiana benthamiana plants. The N. benthamiana plants used for inoculations were kept in the dark for at least 16 hours before inoculations of lower leaves. Following inoculations, plants were watered and returned to greenhouse conditions until harvested.

The inoculated N. benthamiana leaves were harvested fourteen days after the initial inoculation of the infectious clone. Leaves were homogenized by grinding with a mortar and pestle in a 50 mM phosphate buffer (pH 7.2). The homogenate was used for propagation of the virus infection, or the sample was lyophilized and stored with desiccant at 4° C. until use.

### Example 11

### Detection of SEC1-12C and SEC1-12C/SEA in N. benthamiana

Western blot analysis was used to examine the expression level of he rTMV constructs in N. benthamiana using hyper-immune sera generated against bacterial SEC1 or SEA. Briefly, soluble plant proteins were extracted from leaves (fresh or frozen at −80° C.) by homogenizing with a chilled mortar and pestle in cold phosphate buffered saline-Tween-20 (PBST) (50 mM PO₄⁻, 140 mM NaCl, 0.05% Tween-20, pH 7.4) at a ratio of 0.5 ml PBST/1 g of tissue. Plant debris was removed by centrifugation at 5,000×g for five minutes at 4° C. and the soluble extract was removed. Samples were prepared for SDS-PAGE by mixing with 5×sample buffer (50 mM Tris-HCl pH 6.8, 100 mM 2-mercaptoethanol, 2% SDS, 0.1% bromophenol blue and 10% glycerol) and heating at 100° C. for five minutes. Proteins were separated by 12% SDS-PAGE using a Mini-Protein II slab gel apparatus (Bio-Rad, Hercules, Calif.) and transferred to a nitrocellulose membrane (0.1 μm pore size) (Schleicher & Schuell, Keene, N.H.) with the Mini-Protein II Trans Blot Apparatus (Bio-Rad). Buffer systems used for electrophoresis have been previously described (Sambrook et al (1989) Molecular Cloning: A Laboratory Manual., 2nd ed. Cold Spring Harbor Laboratory Press, Cold Spring Harbor, N.Y.). After transfer, non-specific protein binding sites were blocked by incubating the membranes in 5% nonfat dry milk in PBST (PBSTM) for one hour at room temperature. Nitrocellulose membranes were washed in PBST before incubation for two hours at room temperature with hyper-immune serum in 1% PBSTM. At this point, 5% soluble plant extract from non-infected plant tissue was added to reduce the nonspecific binding of the hyper-immune sera to the plant proteins. After three washes in PBST, the membrane was incubated with an alkaline phosphatase-conjugated species-specific anti-immunoglobulin (Sigma) in 1% PBSTM for two hours at room temperature. The membrane was washed in once in PBST followed by three washes in TBS (10 mM Tris-HCl, 140 mM NaCl, pH 7.5) before the antigen/antibody complexes were visualized by the addition of Western Blue™ substrate for alkaline phosphatase Promega). The reaction was stopped with several washes of PBST.

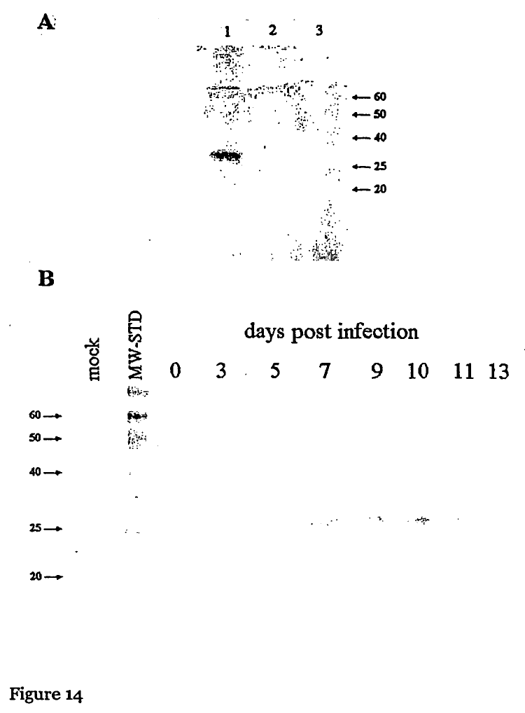

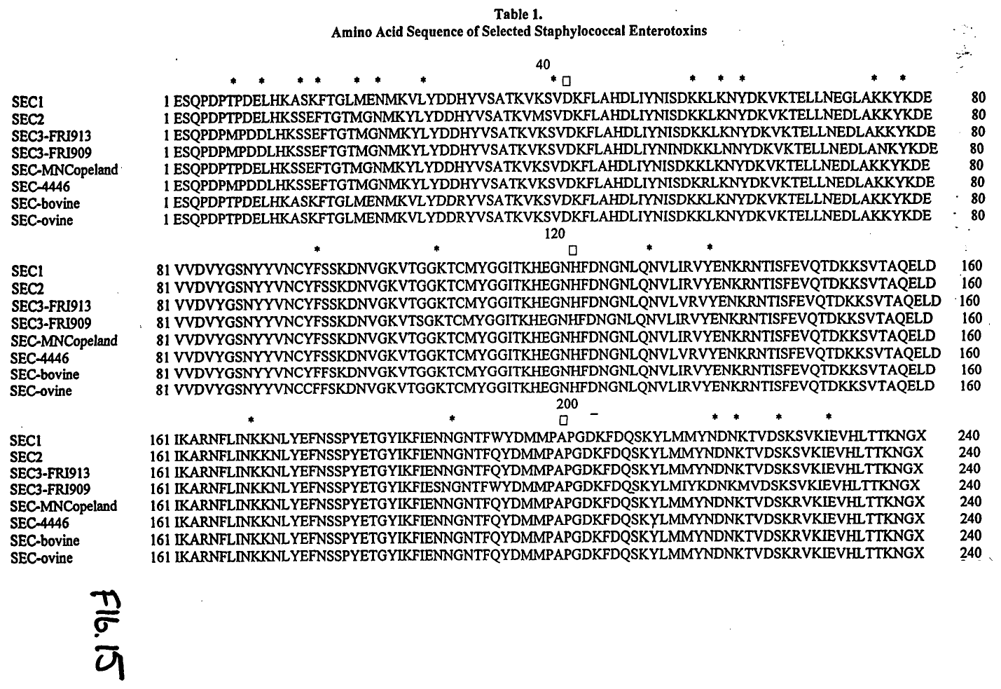

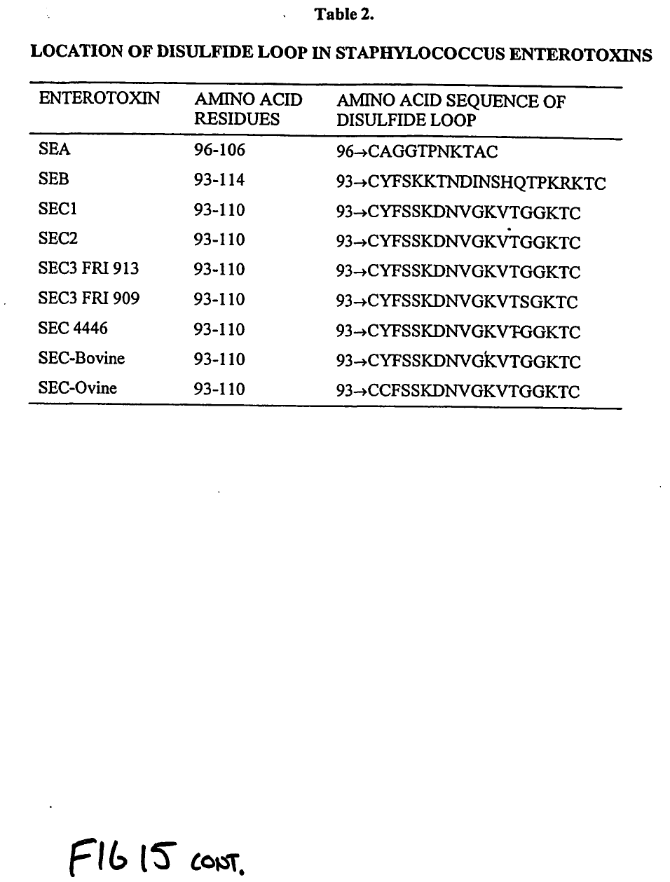

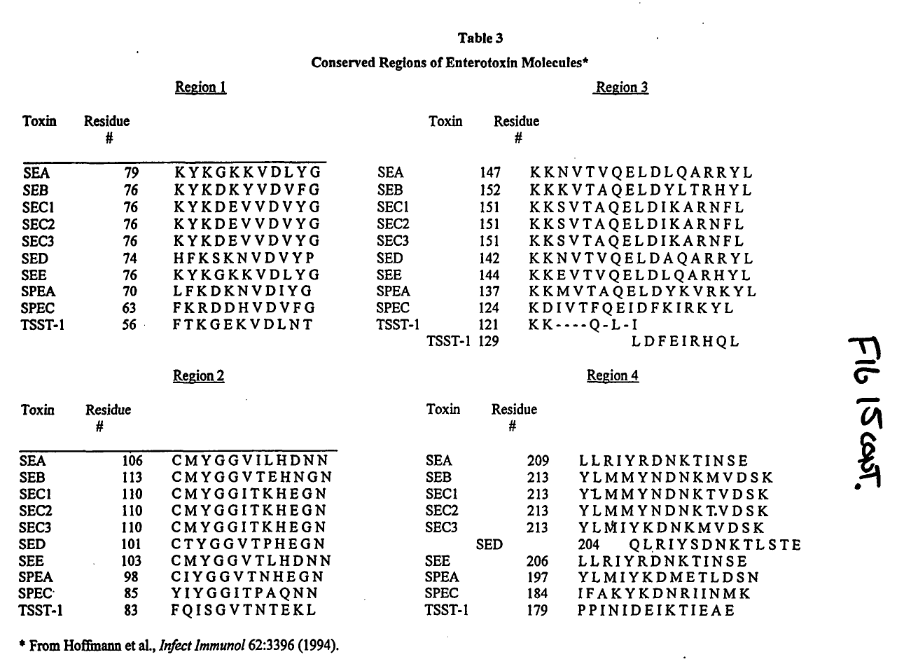

The immunoblot assay indicated that SEC1-12C and SEC1-12C/SEA are expressed in virally infected N. benthamiana leaves. FIG. 14A shows the expression of SEC1-12C and SEC1-12C/SEA in the soluble leaf extract of N. benthamiana plants at day 10 pi compared to plants infected with TMV-30B or uninfected control plants. While the plant produced SEC1-12C/SEA was the expected molecular weight (30 kDa), the observed molecular weight of SEC1-12C was larger (39 kDa) than expected. Both of the SE mutants expressed in N. benthamiana were unaffected by proteolytic degradation as detected by immunoblot with polyclonal antisera. The yield of SEC1-12C and SEC1-12C/SEA expressed in leaf tissue was estimated using immunoblot analysis (data not shown).

### Example 12

### In vitro Infection of Chenopodium quinoa

The in vitro process described in Example 10 was used to infect other plant species with SEC1-12C and SEC1-12C/SEA [+ a control?], including Chenopodium quinoa.

### Example 13

### Detection of SEC1-12C and SEC1-12C/SEA in C. quinoa

Immunoblot analysis was used to examine the expression level of the rTMV constructs in other plant species, using essentially the same protocol as described in Example 11.

The expression of SEC1-12C and SEC1-12C/SEA was monitored over the duration of viral infection in C. quinoa. Leaf tissue was collected from infected plants at days 0, 3, 5, 7, 9, 10, 11, and 13 post inoculation and stored at −80° C. until the conclusion of the experiment. Additionally, the corresponding virus symptoms on each day were recorded.

The expression of both SEC1-12C and SEC1-12C/SEA was detectable by immunoblot analysis in the soluble leaf extract of C. quinoa. Both rTMV constructs were expressed in Chenopodium quinoa, in particular, high levels of SE mutants were expressed in the leaves of these infected plants. SEC1-12C expression in plants at day 10 pi was compared to plants infected with TMV-30B and uninfected control plants by immunoblot analysis. The plant-produced SE's were the expected molecular weight (30 kDa) and proteolytic degradation or post-translational modification products were not observed.

The time dependant accumulation of the plant-produced SEC1-12C in C. quinoa was also examined. Beginning at day 5 pi, recombinant SEC1-12C could be detected. The protein continued to accumulate until days 9-10 pi, at which point, the level of SEC1-12C began to decline. By day 13 pi, SEC1-12C levels were undetectable. This corresponded to the visual yellowing and overall deterioration of the leaves. Visual observations of the infected leaves throughout the time course experiment revealed that the appearance of yellow local lesions occurred by day 10 pi on the infected leaves. This corresponded with the appearance of local lesions on plants infected with 30B.GFP. Additionally, these lesions, like 30B.GFP lesions, could not be used to propagate more infection through back inoculations. Taken together, these findings show that sufficient levels of stable SEC1-12C can be rapidly expressed in C. quinoa for immunizations.

### Example 14

### Plant Host-range Analysis

Previous studies have reported using N. benthamiana as the host for TMV-30B (Shivprasad et al. (1999) Virology. 255(2):312-23; Wigdorovitz et al. (1999) Virology. 264(1):85-91). In this Example, several other hosts were examined for the ability of the virus to propagate, and for the expression of the GFP reporter gene. The determination of recombinant virus host range was accomplished by inoculations of the 30B.GFP virus. Since some plants that produce edible tissues and fruits are naturally infected with TMV, the host range and foreign gene expression levels of the modified viruses in a variety of plants was examined (i.e., N. benthamiana, C. quinoa, S. tuberosum, L. esculentum and several Nicotiana sp.).

Lower leaves of the host plants were inoculated from virus stocks and visually monitored for the ability to cause a local or systemic infection. When necessary, back-inoculations onto known susceptible hosts were used to help determine the presence of infection. At multiple time points after 30B.GFP inoculation, long wave UV illumination was used to access the local or systemic reporter gene expression. Relative fluorescence was recorded and used to guide in the selection of suitable hosts for SE expression experiments.

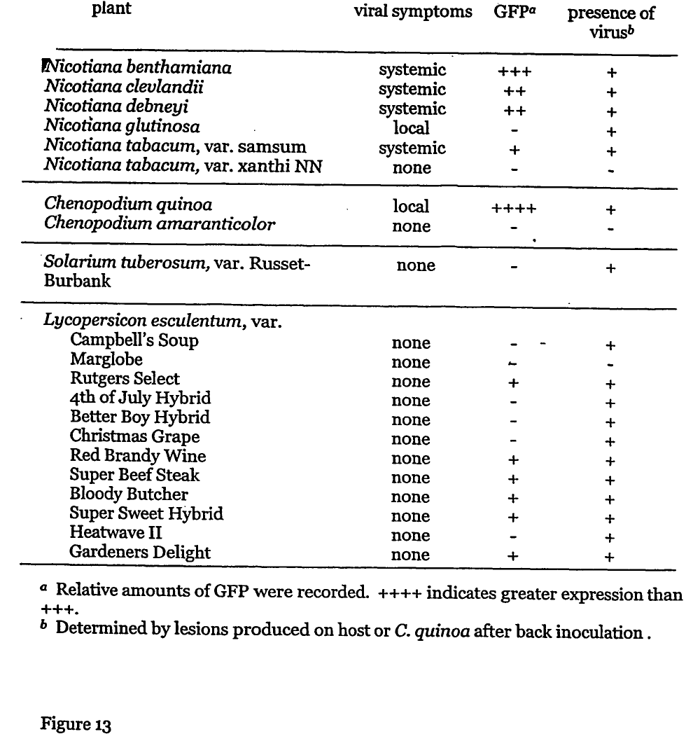

Recombinant TMV infectious clones successfully propagated viral infections of TMV-30B, 30B.GFP, and 30B.SEC1-12C. FIG. 13 shows the host range and foreign gene expression of 30B.GFP in these plants. GFP reporter gene expression in Solarium tuberosum, Lycopersicon esculentum and several Nicotiana sp. was low or not detectable (FIG. 13). However, two plants produced high levels of the recombinant GFP: N. benthamiana and C. quinoa. However, systemic GFP expression in N. benthamiana was reduced when compared to the original infection. GFP expression C. quinoa was slightly higher then GFP levels in N. benthamiana. Additionally, C. quinoa plants infected with 30B.GFP displayed none of the classic systemic viral symptoms seen in the N. benthamiana infections. Instead, yellow local lesions occurred by day 10 pi on only the infected leaves. These lesions could not be used to propagate more infection.

Importantly, C. quinoa leaves can be eaten as a vegetable (Simmonds, N. W (1984) Quinoa and relatives. Chenopodium spp. (Chenopodiaceae), p. 29-30. In N. W. Simmonds (ed.), Evolution of Crop Plants. Longman Inc., New York), suggesting that C. quinoa may be a better host than N. benthamiana for the expression of plant derived ‘edible’ vaccines. Additionally, lesions on infected leaves of C. quinoa could not be used to propagate more infection through back inoculations.

### Example 15

### Time-course Experiments

Time-course experiments showed that maximal expression of the SE mutants in C. quinoa occurred between 7 and 10 days post-inoculation.

### Example 16

### Antigenicity

Antigenticity of the plant-produced rTMV-30B.SEC-12C and rTMV-30B.SEC1-12C/SEA was tested in rabbits by both injection and oral administration. These results demonstrate the efficacy of recombinant plant virus as a feasible expression system for the production of edible bacterial SE toxoid vaccines.

What is claimed is:

1. A modified pyrogenic toxin derived from a native disulfide loop-containing pyrogenic toxin, wherein the modified toxin comprises a disulfide loop containing no more than 10 amino acids.

2. The modified toxin of claim 1 wherein the native disulfide loop-containing pyrogenic toxin is a staphylococcal toxin or a streptococcal toxin.

3. The modified toxin of claim 2 wherein the staphylococcal toxin is a type A, B, C, D, E, G, or H staphylococcal enterotoxin.

4. The modified toxin of claim 1 wherein the disulfide loop region contains no more than 8 amino acid residues.

5. The modified toxin of claim 1 wherein the disulfide loop region contains no more than 3 amino acid residues.

6. The modified toxin of claim 1 wherein the native disulfide loop-containing pyrogenic toxin is a type C staphylococcal enterotoxin.

7. The modified toxin of claim 1 wherein the modification comprises a deletion of between 4 to 18 amino acid residues within the disulfide loop region.

8. The modified toxin of claim 5 wherein the type C staphylococcal enterotoxin is, staphylococcal enterotoxin C1.

9. The modified toxin of claim 8 wherein the staphylococcal enterotoxin is staphylococcal enterotoxin C1, staphylococcal enterotoxin C2, staphylococcal enterotoxin C2, staphylococcal enterotoxin C-MNCopeland, staphylococcal enterotoxin C-4446, staphylococcal enterotoxin C-bovine, staphylococcal enterotoxin C-canine or staphylococcal enterotoxin C-ovine.

10. The modified toxin of claim 1 having an emetic response inducing activity decreased by at least about 100-fold in comparison to a native toxin.

11. The modified toxin of claim 1 having a fever inducing activity decreased by at least about 100-fold in comparison to a native toxin.

12. The modified pyrogenic toxin of claim 1 comprising a N-terminal domain of a first staphylococcal toxin and a C-terminal domain of a second staphylococcal toxin.

13. The modified pyrogenic toxin of claim 1 further comprising an exogenous sequence of between 1 and 30 amino acid residues located within the disulfide loop region.

14. The modified pyrogenic toxin of claim 13 wherein the exogenous sequence comprises a sequence of alanine amino acid residues.

15. An expression vector comprising a nucleic acid sequence encoding a modified pyrogenic toxin according to claim 1.

16. The expression vector of claim 15 comprising a tobacco mosaic virus vector.

17. A host cell transformed with the expression vector of claim 15.

18. The host cell of claim 17 wherein the host cell is a plant cell.

19. The host cell of claim 18 wherein the plant cell is from Nicotiana benthamiana or Chenopodium quinoa.
